(19) United States
(12) Patent Application Publication
(10) Pub. No.: US 2005/0037436 A1
(43) Pub. Date: Feb. 17, 2005

(54) Test systems and the use thereof for identifying and characterizing compounds

(76) Inventors: Georg von Samson-Himmelstjerna, Mellendorf (DE); Achim Harder, Koln (DE); Frank Wunderlich, Neuss (DE); Hans-Peter Schmitt-Wrede, Erkelenz (DE); Beate Saeger, Dusseldorf (DE)

Correspondence Address: JEFFREY M. GREENMAN;VICE PRESIDENT, PATENTS AND LICENSING, BAYER CORPORATION, 400 MORGAN LANE, WEST HAVEN, CT, 06516, US

(21) Appl. No.: 10/363,946

(22) PCT Filed: Aug. 24, 2001
(86) PCT No.: PCT/EP01/09771

(30) Foreign Application Priority Data
Sep. 7, 2000 (DE) 100 44 089.3
Oct. 30, 2000 (DE) 100 53 785.5

(51) Int. Cl. G01N 33/53; G01N 33/567

(52) U.S. Cl. 435/7.2

(57) ABSTRACT

The invention relates to test systems which are based on transmembrane receptors from helminths and arthropods, and to the use thereof for identifying and characterizing substances which act on helminths, arthropods or which act on the calcium balance of organisms and/or cells. The invention furthermore relates to the use of a specific ligand in this test system and to the use thereof as anthelmintic or arthropodicidal active substance.

The present invention relates to test systems which are based on transmembrane receptors from helminths and arthropods, and to the use thereof for identifying and characterizing substances which act on helminths, arthropods or which act on the calcium balance of organisms host cells. The invention furthermore relates to the use of a specific ligand in this test system and to the use thereof as anthelmintic or arthropodicidal active substance.

Parasitic helminths and arthropods represent a considerable health problem for humans and animals. In the agricultural sector alone, the cost for controlling or preventing the damage caused by such parasites around the world amounts annually to more than 7 billion DM (1997 figure). A large number of active substances from a number of classes of active substances are available for the treatment of the endoparasitoses mainly caused by helminths, and of the ectoparasitoses primarily caused by arthropods. During the last two decades there has been an increase in the importance in particular of active substances which simultaneously show a very good action on a plurality of parasite species within a phylum (e.g. helminths), or even extend over various zoological phyla (e.g. helminths and insects). The former include inter alia broad-spectrum anthelmintics such as, for example, the benzimidazoles or the imidazothiazoles, while the macrocyclic lactones are included among the latter.

The last time a novel, broadly active group of active substances was launched on the market was about two decades ago, with the macrocyclic lactones. However, a large number of endoparasite and ectoparasite species have now developed resistance to individual classes, and in some cases simultaneously to a variety of classes, of active substances. Hence there is a continuing and increasingly urgent need for the development of novel substances with antiparasitic activity.

The few promising novel groups of active substances being worked on at present include the cyclic depsipeptides which are the subject of extensive patent (WO 93/19053, EP 626 376, WO 94/19334, WO 95/07272, EP 626 375, EP 657 171, EP 657 172, EP 657 173) and research activities (Conder et al. 1995; Martin et al. 1996; Sasaki et al. 1992; Terada M 1992; Samson-Himmelstjerna et al. 2000).

A number of studies to elucidate the mechanism of action of one representative of this group of active substances, PF 1022A (cyclo(-D-Lac-L-MeLeu-D-PhLac-L-MeLeu-)₂) have already been described (cf. information on this in DE-A-197 04 024).

These included the identification and characterization of a specific binding protein for cyclic depsipeptides and of the DNA sequence coding for the protein from the sheep parasite Haemonchus contortus (DE-A-197 04 024). Within the scope of the present invention, the functional interaction of the abovementioned protein, called HC110-R, inter alia with the ligand BAY 44-4400 (cyclo(-D-Lac-L-MeLeu-D-p-morpholinyl-PhLac-L-MeLeu-), as a further representative of the cyclic depsipeptides, is described. For this purpose, recombinant eukaryotic cell lines in which HC110-R, based on sequence ID No. 2 described in the earlier application DE-A-197 04 024, is expressed have been constructed.

The present invention is accordingly based in particular on the object of providing, on the basis of transmembrane receptors of helminths and arthropods, preferably from nematodes and acarina, particularly preferably from Trichostrongylidae, very particularly preferably from Haemonchus spp., and especially on the basis of the receptor HC110-R from H. contortus, test systems with a high throughput of test compounds (high throughput screening assays; HTS Assays).

Homologous proteins are regarded as being those proteins which have at least 70% identity, preferably 80% identity, particularly preferably 90% identity, very particularly preferably 95% identity, with a sequence as shown in SEQ ID NO: 2 of the document DE-A-197 04 024, the contents of which are to be expressly included in the present application, over a length of at least 20, preferably at least 25, particularly preferably at least 30 consecutive amino acids and very particularly preferably over the complete lengths thereof.

The degree of identity of the amino acid sequences is preferably determined with the aid of the GAP programme from the GCG programme package, Version 9.1, with standard settings (Devereux et al. 1984).

The object is achieved by providing polypeptides which exercise at least one biological activity of a GPC receptor, and by providing a method for obtaining these polypeptides and by providing methods for identifying compounds with nematicidal and arthropodicidal activity.

Test systems based on recombinant microorganisms have already been used many times for identifying pharmaceutically active substances, inter alia also with use of microorganisms which recombinantly express parasitic genes (Klein and Geary 1997). However, to date, no systems in which parasitic transmembrane receptors are used as recombinant functional proteins in eukaryotic cells as targets have been disclosed. The test systems described in this invention can be used to identify this novel class of receptors in high-throughput-screening (HTS) or in ultra-HTS.

Recombinant expression of receptors from nematodes has ordinarily proved to be difficult. Thus, it has not to date generally been possible to express G-protein-coupled receptors (GPCR) from nematodes in such a way that their functional properties (e.g. sensitivity to inhibitors) correspond to those of natural receptors.

Functional expression of receptors from helminths, in particular nematodes, and from arthropods in eukaryotic systems is of great practical significance, for example in the search for novel anthelmintics or arthropodicides.

The present invention is thus also based on the object of providing a possible way of expressing transmembrane receptors, especially GPC receptors, from nematodes and arthropods and, based on this, to develop a test system which makes it possible to identify novel substances with nematicidal and arthropodicidal activity.

The present invention thus relates in particular to the expression and use of an orphan G-protein-coupled receptor from helminths and arthropods, preferably from nematodes and acarina, particularly preferably from Trichostrongylidae, very particularly preferably from Haemonchus and most preferably from the parasitic nematode H. contortus, as target protein for the efficient search for nematicidal active substances.

This receptor has been identified in a cDNA library which was obtained from the gastrointestinal nematode H. contortus. The cDNA codes for a heptahelical transmembrane protein with a size of 110 kDa, which has been referred to as HC110-R. The protein belongs to the secretin family of G-protein-coupled receptors (GPCR) and shows great similarity with latrophilin (FIG. 5).

The HC110-R Receptor as Target

The GPCR latrophilin was originally isolated from mammalian brain. Latrophilin has a molecular mass of 210 kDa and undergoes post-translational cleavage at residue 18 upstream of the first transmembrane segment, and thus consists of two non-covalently linked subunits. The p120 subunit contains the N-terminal hydrophilic extracellular portion, and the p85 subunit contains the seven transmembrane domains and the intracellular C-terminal region of latrophilin, which is unusually large for GPCRs. Two close homologues have recently been identified, latrophilin-2 and latrophilin-3 (see also FIG. 6). The latter is preferentially, like latrophilin-1 too, expressed in the brain, while latrophilin-2 is expressed ubiquitously with a preference for placenta, kidney, spleen, ovaries, heart and lung of mammals (Ichtchenko et al. 1999; Sugita et al. 1998).

Although HC110-R is only half the size of the 210 kDa latrophilin, the similarity of the sequence also extends to a functional similarity. The endogenous ligand for both receptors is still unknown, but both latrophilin and HC110-R are influenced by the artificial ligand α-LTX (alpha-latrotoxin).

If, for example, HEK-293 cells are transiently transfected with latrophilin, the addition of α-LTX causes influx of external Ca²⁺, as can be shown by means of an experimental design with radioactive ⁴⁵Ca²⁺. α-LTX also causes such a Ca²⁺ influx in HEK-293 cells which have been transiently transfected with HC110-R, which can be observed for example by Ca²⁺ imaging (see also FIGS. 10 and 11).

This Ca²⁺ influx is, however, very complex. If the HC110-R is in the form of a construct with a C-terminal Green Fluorescent Protein (GFP) attachment, it is biphasic, i.e. it shows one change after about 3 and another one after about 22 minutes. If, however, only an N-terminal Myc-His tag is put in front of HC110-R, the main influx is observed only about 2-3 minutes after addition of α-LTX. The reason for this is not yet known, but the response to the α-LTX addition is specific, as shown by the subsequent statements:
1. The Ca²⁺ influx is not observable in untransfected cells and in cells transiently transfected with a mouse β₂-adrenergenic receptor.
2. The changes in [Ca²⁺]ᵢ are dependent on the α-LTX dose (FIG. 10).
3. The biphasic change requires influx of Ca²⁺, which takes place through Ca²⁺ channels which can be blocked by Cd 2, especially those of the L type, as is evident from their sensitivity to nifedipine.

The exact mechanism of the interaction of α-LTX with HC110-R and the transduction of signals still requires explanation. It has been possible to show for the example of latrophilin-1 that only a single transmembrane domain is necessary for the Ca²⁺ influx caused by α-LTX into HEK-293 cells (Kraspernov et al. 1999).

The fact that nifedipine blocks the effect of α-LTX in the system described here shows that the system is also suitable for identifying novel specific calcium channel blockers. Nifedipine belongs to a class of calcium channel agonists and antagonists which are classified according to their binding to a specific site of a calcium channel, e.g. on the basis of their binding to L-type channels. The principal three classes of calcium channel blockers (L type) are benzylacetonitriles (e.g. verapamil, WO 91/02497), benzothiazepinones (e.g. diltiazem) and 1,4-dihydropyridine derivatives such as nifedipine, nivaldipine, nimodipine, nicardipine, isradipine, amlodipine, nitrendipine, felodipine or nisoldipine (Bacon et al. 1989; WO 90/09792). Another class of L-type calcium channel blockers comprises 1,3-diphosphonates, e.g. belfosodil.

This invention therefore also relates to the use of α-LTX-binding transmembrane receptors for identifying novel calcium channel blockers. Heptahelical transmembrane receptors are particularly preferably used for this purpose, and they are particularly preferably G-protein-binding receptors. The use of transmembrane receptors of the secretin family is very particularly preferred. Most preference is given to the use of the HC110-R receptor as shown in SEQ ID. NO: 2, and receptor proteins which are 70%, preferably 80%, particularly preferably 90% and very particularly preferably 95% homologous thereto.

The available data also show that HC110-R is a target for the novel anthelmintic depsipeptide BAY44-4400. BAY44-4400 interferes with signal transmission by α-LTX in HEK-293 cells which have been transfected with HC110-R (FIG. 12). This interference may derive from the ionophoric activity of BAY 44-4400, as is typical of other depsipeptides, e.g. beauvericin, enniatin etc. (Geβner et al. 1996). However, BAY44-4400 causes no changes in untransfected cells. In addition, BAY44-4400 does not interfere with the isoproterenol-induced signal transmission through HEK-293 cells transfected with the mouse β₂-adrenergic receptor. In addition, BAY44-4400 acts as antagonist of α-LTX, while BAY44-4400 alone does not influence the Ca²⁺ concentration in HC110-R-transfected HEK-293 cells.

The HC110-R Protein

The membrane protein described in DE 197 04 024 A1 was identified as a possible target protein for the anthelmintic active substance PF1022A. Possible target proteins were identified by preparing a γZAPII cDNA expression library of the parasitic nematode Haemonchus contortus, and screening the cDNA library using a conjugate of PF1022A and KLH (keyhole limpet hemocyanin), PF1022A-KLH, and polyclonal antibodies against this conjugate (see Example 1). Examination of about 1.5×10⁶ non-amplified recombinant clones revealed a cDNA clone with a length of 3036 bp which hybridized with a 3.6 kb mRNA from H. contortus (see also Example 2). The RACE PCR technique was used to complete the 3′ and 5′ ends (Example 4). Finally, a cDNA with a length of 3539 bp was obtained and was referred to as HC110-R. This cDNA codes for 986 amino acids (10 kDa), with the open reading frame starting with ATG¹⁰⁰ (FIG. 2). The start codon is surrounded by a Kozak sequence for optimal initiation of translation. The derived molecular weight of the protein was confirmed by in vitro translation of HC110-R RNA transcribed in vitro.

The amino acid sequence of the HC110-R protein shows some particular features. The extracellular N-terminal part of the protein consists of a total of 535 residues. The N terminus comprises a signal peptide with a length of 21 amino acids and a cleavage site at position 18. This is followed by a lectin-like sequence (AA 22-125) and a so-called “Thr stretch” (AA 128-147) which is interrupted only by a serine in position 144. Downstream of the “Thr stretch” there is a cysteine-rich motif with the structure CX₉WX₁₂CX₉WXCX₅WX₉CX₃W (AA 166-221). In addition, the HC110-R protein contains seven hydrophobic α-helical transmembrane domains between residues 536 and 772. Directly upstream from the transmembrane region there is a further 4-Cys region with the structure CXWWX₆WX₄CX₁₁CXC (AA 478-524). In the transmembrane region there are three extracellular loops comprising the residues 587-597, 654-673 and 743-749 and three intracellular loops (position 559-569, 627-636, 696-724). The C terminus is 214 residues long (24 kDa) and contains a proline-rich part (AA 845-861) and a PEST region (AA 915-933). Finally, there are also three putative N-glycosylation sites at residues 26, 499 and 862, and 14 putative phosphorylation sites in the derived intracellular domains (see also FIG. 6).

Analysis of databases revealed 48% identity and 76% similarity of the HC110-R proteins with an unknown transmembrane protein (1014 AA) which was derived from the genomic clone B0457 (GenBank™ Accession Number Z54306) of the nematode Caenorhabditis elegans (see also FIG. 3).

The term “identity” as used herein describes the number of sequence positions which are identical in a so-called alignment. It is stated as a percentage of the alignment length.

The term “similarity” as used herein describes the similarity of sequences on the basis of a similarity metric, that is to say a measure of how similar for example a valine is to be assumed to be to a threonine or to a leucine.

The term “homology” as used herein means evolutionary relationship, that is to say if two homologous proteins have developed from a common precursor sequence. The term does not necessarily have anything to do with identity or similarity, apart from the fact that homologous sequences are usually more similar, or have more identical positions in an alignment than do non-homologous sequences.

Comparison of the two sequences shows features common to both proteins, for example the lectin-like sequence, the “Thr stretch”, the Cys motifs and the PEST sequence. The greatest identity is to be found in the transmembrane region (62%), whereas the identity is found to be less pronounced in the N-terminal (44%) and C-terminal (50%) region.

In addition, the HC110-R protein has 20-30% identity with heptahelical G-protein coupled transmembrane receptors (GPCR), especially with the secretin subfamily. Comparisons of seven transmembrane domains show a high degree of identity and similarity in relation to structure and sequence between various GPCRs of the secretin subfamily and HC110-R (FIG. 4).

Latrophilin, a member of the secretin subfamily, from mammals such as humans (GenBank™ Accession No.: E1360690), cattle (e.g. G416021, G416053 and G4185804), and rats (U78105 or U72487) show somewhat greater identity (31%) with HC110-R than do other secretin receptors. In particular, the transmembrane regions show an identity of 45-48%. HC110-R and the rat latrophilin-1 GPCR (U78105) of 1466 amino acids display common features, e.g. the lectin domain, the cysteine-rich region and a conserved 4-Cys motif in front of the transmembrane region. The latter was recently proposed to be proteolysis site of latrophilin and other large secretin GPCRs. By contrast, the N terminus of HC110-R does not contain the olfactomedin region and the Pro/Thr region of latrophilin, while latrophilin in turn does not have the “Thr stretch” of HC110-R.

The present application therefore also relates to the use of G-protein-coupled transmembrane receptors with seven transmembrane domains from helminths for identifying substances with anthelmintic activity. Preference is given according to the invention to the use of transmembrane receptors which can be assigned to the secretin subfamily.

Cellular Localization

Transient transfection experiments were carried out with an HC110-R-GFP fusion protein in various mammalian cell lines, for example COS-7 cells or HEK-293 cells (FIG. 7). Heterologous expression was chosen because no H. contortus cell lines have yet been established.

The green fluorescent protein (GFP) from the pacific jellyfish Aequorea victoria can be used for localizing proteins in living cells (see FIG. 8). GFP is used at the cellular level as an in vivo reporter in order to indicate the frequency of a transient or stable transfection and at the subcellular level for localizing proteins. Wild-type GFP is a 27 kD monomer composed of 238 amino acids which emits green light with a maximum of 509 nm after excitation with UV light (360-400 nm; max. at 395 nm) or blue light (440-480 nm; max. at 475 nm), without requiring exogenous substrates or cofactors for this (Chalfie et al. 1994). GFP can thus be detected in vivo directly by fluorescence microscopy, and its fluorescence characteristics are essentially unchanged even when part of a fusion protein. EGFP (enhanced GFP) is a genetic variant of the wild-type GFP and is employed for the transfection of mammalian cells (Yang et al. 1996). The excitation maximum of EGFP has been shifted to only one peak at 490 nm by replacing Ser⁶⁵ by Thr. The vectors pEGFP-1 (GenBank Accession No.: U55763) and pEGFP-N3 (GenBank Accession No.: U55762) [Clontech, Palo Alto, Calif., U.S.A.] express EGFP under the control of the strong constitutive CMV promoter and can be used to fuse other proteins respectively to the N and C terminus of EGFP.

Stable cell lines have the advantage over transient expression that every cell permanently expresses the desired protein, and isolation of the protein is possible after localization. In order to detect the protein by fluorescence microscopy or with the aid of a Western blot, an antibody against the desired protein is required, or it is appropriate to choose an expression vector which, for example, fuses a Myc or His tag C-terminally to the actual protein in the correct reading frame, against which there are then antibodies which can be purchased, in most cases even monoclonal ones.

The present invention likewise relates to cells which make stable expression of the HC110-R receptor, and proteins homologous thereto, possible.

Effect of Alpha-Latrotoxin on the HC110-R Protein

To further substantiate the similarity between HC110-R and latrophilin, and to examine the functionality of the recombinantly expressed HC110-R, HEK-293 cells were transiently and stably transfected with HC110-R-GFP fusion protein and stimulated with alpha-latrotoxin (a-LTR).

Alpha-latrotoxin is a presynaptic neurotoxin which can be isolated from black widow (Latrodectus mactans) venom. It is known for its toxicity for the central nervous system of vertebrates, where it induces the depolarization of neurons through increasing [Ca²⁺]ᵢ and stimulating uncontrolled exocytosis of neurotransmitters. Thus, it has also been disclosed that the effect of α-latrotoxin is mediated at least in part by latrophilin. It is moreover assumed that the toxicity of α-latrotoxin derives from its ability to interact with receptors which are coupled to GTP-binding protein (GPCR). These receptors normally mediate the effect of endogenous hormones or neuropeptides (Holz and Habener 1998).

In the present invention, the so-called Ca²⁺ imaging technique was used in order to test the response of transfected HEK-293 cells to α-LTX, by determining the change in the Ca²⁺ present in the cell [Ca²⁺]ᵢ (see Example 24 and FIGS. 9, 10 and 11).

α-LTX causes a biphasic increase in [Ca²⁺]ᵢ. At a concentration of 75 nM, α-LTX induces initially a very small increase of only 5±0.2 nM 2 minutes after α-LTX addition and a larger, delayed increase of about 220+14.9 nM Ca²⁺ after 22 minutes. As the α-LTX concentration increases (7.5 nM-120 nM), the first increase becomes larger, while the second increase diminishes. At an α-LTX concentration of 120 nM, the first increase reaches values of about 135±13.6 nM Ca²⁺, while the second increase falls to a value of 50+7.1 nM Ca²⁺. The same profile on use of 90 nM and 120 nM α-LTX indicates saturation.

Transfection of HEK-293 cells with an N-terminal GFP-tagged HC110-R construct and stimulation of them with 75 nM α-LTX results in a slightly reduced 2nd peak. Finally, an only slightly diminished response to α-LTX has also been found when the HC110-R protein has been provided with an N-terminal GFP tag. It can therefore be assumed that N- or C-terminal attachment of a GFP tag has a negligible effect on α-LTX binding and subsequent signal transduction by HC110-R.

Cells which had not been transfected or had been only transiently transfected with GFP show no response to α-LTX. If HEK-293 cells are transiently transfected with other G-protein-coupled receptors with C-terminal GFP tags, e.g. the mouse β₂-adrenergic receptor, or the human muscarinergic H1 acetylcholine receptor, the increase in [Ca²⁺]ᵢ caused by α-LTX at a concentration of 75 nM is only small (of about 40+10.4 nM after about 20 minutes) or zero.

The increase in [Ca²⁺]ᵢ caused by α-LTX may lead both to influx of extracellular Ca²⁺ and to efflux of intracellular Ca²⁺. If extracellular Ca²⁺ is removed with 2 mM EGTA before or after the addition of α-LTX, there is only a small increase in the [Ca²⁺]ᵢ of HEK-293 cells which express the HC110-R-GFP fusion protein. It is evident from this that α-LTX primarily causes influx of extracellular Ca²⁺. This Ca²⁺ influx is not based on simple diffusion but takes place with the aid of Ca²⁺ channels in the plasma membrane.

Most of the Ca²⁺ channels involved in the Ca²⁺ influx are those of the L type, because 15 μM nifedipine is sufficient to depress significantly the α-LTX-induced increase in [Ca²⁺]ᵢ. Moreover, the first increase is completely inhibited, and the second increase falls from 267±12.7 nM to 30±5.4 nM.

The stable or transient HEK-293 cell line expressing the HC110-R receptor with a C-terminal Myc/His tag also responds dose-dependently to α-LTX (FIG. 10). 7.5 nM α-LTX are still too little to generate an α-LTX-induced Ca²⁺ influx in this case too.

However, even 25 nM α-LTX are sufficient to generate an increase in [Ca²⁺]ᵢ of 130±38.0 nM after only 2 min. On addition of 75 nM α-LTX there is a Ca²⁺ influx of 296±91.5 nM, which increases greatly likewise 2 min after α-LTX addition and returns to its original Ca²⁺ content only after 27 min. This Ca²⁺ signal resembles the second delayed Ca²⁺ peak of HC110-R-GFP-transfected HEK-293 cells at the same α-LTX concentration (75 nM) in the height of the signal and in its profile, but the response takes place—as also with higher concentrations (90 nM and 120 nM) of HC110-R-GFP-transfected cells—immediately after α-LTX addition.

The invention therefore likewise relates to the use of α-LTX as agonist of transmembrane receptors of the sekretin family from nematodes. α-LTX is preferably used as agonist of the HC110-R receptor as shown in SEQ ID NO: 2, and of receptor proteins which are 70%, preferably 80%, particularly preferably 90% and very particularly preferably 95% homologous thereto.

The present invention likewise relates to the use of α-LTX as a nematicide.

The present invention likewise relates to the use of α-LTX in a method for identifying compounds with nematicidal or arthropodicidal activity, the compounds possibly being active as agonists or antagonists of transmembrane receptors.

The present invention likewise relates to the use of α-LTX in a method for identyfing compounds which block calcium channels.

Effect of BAY 44-4400 on the Action of α-LTX

PF1022A exerts its effect on nematodes at concentrations in the range 100-800 ng/ml, depending on the particular species (Terada, 1992). In order to investigate in each case any possible interaction between PF1022A with HC110-R and the signal transmission mediated by HC110-R, BAY44-4400, which is a readily soluble derivative of PF1022A, was used in the following Ca²⁺ imaging experiments in HEK-293 cells loaded with FURA-2 and transfected with HC110-R-GFP.

At a concentration of 400 ng/ml, neither the very effective nematicide BAY44-4400 nor the virtually ineffective antipode PF1022-001 induces a Ca²⁺ response in HC110-R-GFP-transfected HEK 293 cells, even if the cells had been preincubated with the active substance for 90 minutes. In contrast to this, both substances affect the signal transmission dependent on α-LTX, although to differing extents (FIGS. 12 and 14). In the presence of 4 ng/ml BAY44-4400, α-LTX induces only a small Ca²⁺ increase with a maximum of 44±6.0 nM Ca²⁺ after 14 minutes (FIG. 12). In the presence of PF1022-001, however, α-LTX causes a larger increase of 103±11.5 nM Ca²⁺ after 6 minutes (FIG. 10B). In another approach, the cells were preincubated with either 4 ng/ml or 400 ng/ml BAY44-4400 and PF1022A for 90 minutes, and the active substances were then removed before the cells were stimulated with α-LTX. HEK-293 cells preincubated with PF1022A showed no significant change in their response to α-LTX. Only BAY44-4400 affected the sensitivity of the cells to α-LTX. At a BAY44-4400 concentration of 4 ng/ml, α-LTX induced a Ca²⁺ increase (95±20.5 nM Ca²⁺) 19 minutes before a stable Ca²⁺ level was reached. At 400 ng/ml BAY44-4400, α-LTX caused an increase in the Ca concentration to only about 65+7.5 nM Ca²⁺. In addition, this small increase was shifted by 12 minutes.

In order to verify the specificity of these results, additionally the effect of 1 mM carbachol on the endogenous, natural M1-R was measured in untransfected HEK-293 cells. The presence of 400 ng/ml BAY44-4400 did not change the response of the cells. Nor was the stimulation induced by isoproterenol and arecoline of the endogenous, natural β₂-R or of the nicotinic cholinergic receptor in HEK-293 cells affected by BAY44-4400 in any way.

Finally, within the scope of the present invention, the effect of BAY 44-4400, a more soluble variant of PF1022A, on the HC110-R protein in HEK-293 cells which transiently or stably express an HC110-R protein provided with C-terminal GFP was investigated by Ca²⁺ imaging. On incubation of HEK-293 cells with only 400 ng/ml BAY 44-4400, the cells responded for 50 minutes in no comparable way with a change in the intracellular Ca²⁺ concentration. However, an effect of BAY 44-4400 can be found in the presence of α-LTX. Incubation of cells with BAY 44-4400 for 6 minutes before addition of 75 nM α-LTX reduces the effect of α-LTX on the Ca²⁺ concentration. The first small increase in [Ca²⁺]ᵢ disappears and the second is reduced by 85% 15 minutes after addition of α-LTX. The response to α-LTX is even stronger if the cells are preincubated with BAY 44-4400 for 60 minutes before the cells are treated with FURA for 30 minutes. There is a complete disappearance of the first increase in Ca²⁺ and an only very small second increase of 70 nM [Ca²⁺]ᵢ 30 minutes after addition of 75 nM α-LTX.

In order to show the specific effect of BAY 44-4400 on the action of α-LTX, two control experiments were carried out. Firstly, HEK-293 cells were transiently or stably transfected with a mouse β₂-adrenergic receptor provided with a C-terminal GFP tag, and isoproterenol was used as ligand.

In fact, isoproterenol also causes a significant Ca²⁺ response: immediately after the addition of isoproterenol there is only one increase in Ca²⁺. This increase is, however, unaffected by BAY 44-4400.

In addition, the specificity of the interaction between BAY 44-4400 and HC110-R is shown with the aid of the optical antipode which occupies the site of BAY 44-4400 as ligand. After preincubation of the cells with 400 ng/ml of a PF1022A derivative with 100-fold weaker anthalmintic activity, PF1022-001: cyclo(-L-Lac-D-MeLeu-L-PhLac-D-Me-Leu-)₂ (also referred to as optical antipode) for 90 minutes there was an increase in Ca²⁺ of only 110 nM 6 minutes after addition of 75 nM α-LTX, that is to say a reduction by 59%. When 4 ng/ml of the optical antipode are added 6 minutes before the addition of 75 nM α-LTX there is an increase of 67 nM [Ca²⁺]ᵢ immediately after addition of the α-LTX. If the concentration of the optical antipode increases there is a reduction in the increase to 20 nM Ca²⁺.

The term “polypeptide” as used herein refers both to short amino acid chains, which are usually referred to as peptides, oligopeptides or oligomers, and to longer amino acid chains, which are usually referred to as proteins. It encompasses amino acid chains which may be modified either by natural processes, such as post-translational processing, or by chemical methods, which are state of the art. Such modifications may occur at various sites and more than once in a polypeptide, such as, for example, on the peptide backbone, on the amino acid side chain, at the amino terminus host carboxy terminus. They encompass for example acetylations, acylations, ADP ribosylations, amidations, covalent linkages with flavins, haem portions, nucleotides or nucleotide derivatives, lipids or lipid derivatives or phosphatidylinositol, cyclizations, disulfide bridge formations, demethylations, cystine formations, formylations, gamma-carboxylations, glycosylations, hydroxylations, iodinations, methylations, myristoylations, oxidations, proteolytic processings, phosphorylations, selenoylations and tRNA-mediated additions of amino acids.

The polypeptides may according to the invention be used in the form of “mature” proteins or as parts of larger proteins, e.g. as fusion proteins. They may furthermore have secretions or “leader” sequences, pro-sequences, sequences which make simple purification possible, such as multiple histidine residues, or additional stabilizing amino acids.

Homologous proteins or polypeptides are regarded as being those proteins or polypeptides which have at least 70% identity, preferably 80% identity, particularly preferably 90% identity, very particularly preferably 95% identity, with a sequence as shown in SEQ ID NO: 2 of the document DE-A-197 04 024, the contents of which are to be expressly included in the present application, over a length of at least 20, preferably at least 25, particularly preferably at least 30 consecutive amino acids and very particularly preferably over the complete lengths thereof.

The polypeptides need not, for their use according to the invention, represent complete receptors, but may also be only fragments thereof as long as they still have at least the biological activity of the complete receptors. It is moreover unnecessary for the polypeptides to be derivable from transmembrane receptors of H. contortus. Polypeptides which correspond to transmembrane receptors of other species of helminths or even arthropods, or fragments thereof which are still able to exercise the biological activity of these receptors, are also regarded as being according to the invention.

The polypeptides may, for their use according to the invention, have deletions or amino acid substitutions compared with the corresponding region of naturally occurring GPC receptors, as long as they still exercise at least one biological activity of the complete receptors. Conservative substitutions are preferred. Such conservative substitutions encompass variations where one amino acid is replaced by another amino acid from the following group:
1. Small aliphatic, nonpolar or slightly polar residues: Ala, Ser, Thr, per and Gly;
2. polar, negatively charged residues and amides thereof: Asp, Asn, Glu and Gln;
3. polar, positively charged residues: His, Arg and Lys;
4. large aliphatic, nonpolar residues: Met, Leu, Ile, Val and Cys; and
5. aromatic residues: Phe, Tyr and Trp.

The following list shows preferred conservative substitutions:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

The sequences described above and in SEQ ID NO: 1 and SEQ ID NO: 3 in DE-A-197 04 024 may additionally be used for finding genes which code for polypeptides which are involved in the structure of functionally similar transmembrane receptors in helminths or arthropods. Functionally similar receptors mean according to the present invention receptors which encompass polypeptides which, although they differ in the amino acid sequence from the polypeptides described herein, have essentially the same biological function.

The term “essentially the same biological function” as used herein means involvement in the structure of G-protein-coupled heptahelical transmembrane receptors capable of functioning, particularly such receptors of the sekretin subfamily, or a function corresponding to the HC110-R receptor from H. contortus. Such a function also includes the properties described above of the receptor, such as the sensitivity to α-LTX as agonist or nifedipine as antagonist of α-LTX.

The term “hybridize” as used herein describes the process in which a single-stranded nucleic acid molecule undergoes base pairing with a complementary strand. It is possible in this way, starting from the sequence information disclosed herein, to isolate for example DNA fragments from other nematodes than H. contortus, and from arthropods, which code for polypeptides having the biological activity of GPC receptors.

In relation to a suitable probe, the amino-terminal and carboxy-terminal cDNA sections are preferred. The hybridization conditions are chosen so that it is also possible to detect less similar sequences from other organisms. The hybridization conditions with reduced stringency may be as follows, for example: hybridization is carried out in 6×SSC/0% formamide as hybridization solution at between 40 and 55° C. The conditions for the specific 2nd washing step must be tested, e.g. initially 2×SSC at 50° C., then estimation of the signal intensities. The washing conditions are then modified.

Suitable hybridization conditions are indicated by way of example below:
Hybridization solution: 6×SSC/0% formamide, preferred hybridization solution: 6×SSC/25% formamide
Hybridization temperature: 34° C., preferred hybridization temperature: 42° C.
1st washing step: 2×SSC at 40° C.
2nd washing step: 2×SSC at 45° C.; preferred 2nd washing step: 0.6×SSC at 55° C.;
   particularly preferred 2nd washing step: 0.3×SSC at 65° C.
Hybridization conditions are calculated approximately by the following formula:
   The melting temperature Tm=81.5° C.+16.6 log[c(Na⁺)]+0.41(% G+C))−500/n (Lottspeich and Zorbas 1998).

In this, c is the concentration and n is the length of the hybridizing sequence section in base pairs. The term 500/n is omitted for a sequence of >100 bp. Maximum stringency washing is at a temperature of 5-15° C. below Tm and an ionic strength of 15 mM Na⁺ (corresponds to 0.1×SSC). If an RNA probe is used for the hybridization, the melting point is 10-15° C. higher.

The polypeptides used in the method according to the invention for identifying compounds with nematicidal and arthropodicidal activity are encoded by the nucleic acids described in SEQ ID NO: 1 and SEQ ID NO: 3 in DE-A-197 04 024.

Also included in the use according to the invention are nucleic acids which have at least 70% identity, preferably 80% identity, particularly preferably 90% identity, very particularly preferably 95% identity with a sequence as shown in SEQ ID NO: 1 or SEQ ID NO: 3 over a length of at least 20, preferably at least 100, particularly preferably at least 500 consecutive nucleotides, and very particularly preferably over the entire length thereof.

The nucleic acid according to the invention can likewise be used for producing transgenic invertebrates. The latter can be employed in test systems which are based on an expression of the receptors according to the invention or variants thereof which differs from the wild type. Also included in this are all transgenic invertebrates for which modification of other genes or gene control sequences (e.g. promoters) results in a change in the expression of the receptors according to the invention or variants thereof.

Production of the transgenic invertebrates takes place for example in Drosophila melanogaster by P-element-mediated gene transfer or in Caenorhabditis elegans by transposon-mediated gene transfer (e.g. by Tc1, Plasterk 1996).

The invention thus also relates to transgenic invertebrates which comprise at least one of the nucleic acids according to the invention, preferably transgenic invertebrates of the species Drosophila melanogaster or Caenorhabditis elegans, and the transgenic progeny thereof. The transgenic invertebrates preferably comprise the receptors according to the invention in a form which differs from the wild type.

The nucleic acid of the invention can be produced in a conventional way. For example, complete chemical synthesis of the nucleic acid molecule is possible. It is also possible for only short pieces of the sequence according to the invention to be synthesized chemically and for such oligonucleotides to be labelled radioactively or with a fluorescent dye. The labelled oligonucleotides can be used to screen cDNA libraries produced starting from nematode mRNA or insect mRNA. Clones which hybridize to the labelled oligonucleotides are selected for isolation of the relevant DNA. After characterization of the isolated DNA, the nucleic acid of the invention is obtained in a simple manner.

The nucleic acid according to the invention can also be produced by PCR methods using chemically synthesized oligonucleotides.

The term “oligonucleotide(s)” as used herein means DNA molecules which consist of 10 to 50 nucleotides, preferably 15 to 30 nucleotides. They are, for example, chemically synthesized and can be used as probes.

The nucleic acid according to the invention can be used for the isolation and characterization of the regulatory regions which occur naturally in the vicinity of the coding region. The present invention thus likewise relates to such regulatory regions.

The methods according to the invention likewise make use of vectors which comprise a nucleic acid to be used according to the invention or a DNA construct to be used according to the invention. Vectors which can be used are all phages, plasmids, phagemids, phasmids, cosmids, YACs, BACs, artificial chromosomes or particles suitable for particle bombardment, which are used in molecular biology laboratories.

Preferred vectors are pBIN and its derivatives for plant cells, pFL61 for yeast cells, pBLUESCRIPT vectors for bacterial cells, lamdaZAP (from Stratagene) for phages.

Various vectors have been used and a plurality of constructs have been produced within the scope of the present invention. The present invention likewise relates to the vectors used for transient or stable transformation of cell lines and for stable expression of the HC110-R receptor.

EGFP constructs which lead to the expression of fusion proteins with an N-terminal EGFP tag (EGFP-HC110-R) or a C-terminal EGFP tag (HC110-R-EGFP) were produced for transient expression in eukaryotic cells. The present invention likewise relates to the polypeptides encoded by these vectors and the conventional GFP vectors, and red-shift and blue-shift variants.

A further construct, the vector pMyc6×His, was used for stable expression, and the present invention likewise relates thereto. The vector pMyc6×His is derived from the vector pSecTagA [Invitrogen, Leek, NL] by double digestion with the restriction enzymes NhiI and SfiI, followed by blunting of the ends and religation of the vector.

The present invention also relates to host cells which comprise a nucleic acid to be used according to the invention or a vector to be used according to the invention. This invention relates in particular to a stably transformed HEK-293 cell line with HC110-R-Myc/His which has been deposited under the number DSM ACC2464 at the international depositary authority DSMZ-Deutsche Sammlung von Microorganismen und Zellkulturen GmbH, Mascheroder Weg lb in 38 124 Braunschweig.

The present invention likewise relates to host cells which comprise a nucleic acid to be used according to the invention or a vector to be used according to the invention, and a vector which enables the cells to express aequorin, a luminescent protein which 2+emits light in the presence of Ca²⁺. Corresponding host cells make it possible to follow the change in the Ca²⁺ concentration, and thus the effect of substances for example on HC110-R, with the aid of the aequorin indicator. The invention relates in particular to the stably transformed HEK-293 cell line with HC110-R-Myc/His which is capable of expressing aequorin and has been deposited under the number DSM ACC2465 at the international depositary authority DSMZ-Deutsche Sammlung von Microorganismen und Zellkulturen GmbH, Mascheroder Weg 1b in 38 124 Braunschweig.

The term “host cell” as used herein refers to cells which do not naturally contain the nucleic acids according to the invention.

Suitable and preferred host cells are eukaryotic cells such as yeasts, mammalian, amphibian, insect or plant cells. Preferred eukaryotic host cells are HEK-293, Schneider S2, Spodoptera Sf9, Kc, CHO, HepG-2, K1, COS-1, COS-7, HeLa, C127, 3T3 or BHK cells and, in particular, xenopus oocytes, and HEK-293 or COS-7 cells are very particularly suitable.

This invention likewise relates to the use of DNA corresponding to the sequence ID No. 2 described in the application DE-A-197 04 024 for the detection of DNA from helminths, preferably from the phylum Nematoda, particularly preferably from the family Trichostrongylidae, very particularly preferably of the genus Haemonchus and most preferably of the species Haemonchus contortus.

The invention moreover relates to oligonucleotides which correspond to a region of the DNA sequence described above or its complementary strand and are able to hybridize thereto. The invention relates to the use of these oligonucleotides or parts thereof as
a) probes in Northern or Southern blot assays,
b) PCR primers in a diagnostic method for detecting the above-mentioned nematodes, where the DNA of the relevant helminths is specifically amplified with the aid of the primers and of the PCR technique and is then identified.

The invention also relates to a method for detecting helminths, preferably from the phylum Nematoda, particularly preferably from the family Trichostrongylidae, very particularly preferably of the genus Haemonchus and most preferably of the species Haemonchus contortus, where oligonucleotides as described above specifically hybridize to DNA sequences which originate from the said organisms, and which are then amplified with the aid of the PCR technique.

Detection of organisms as mentioned above can take place, for example, by
a) providing an oligonucleotide probe or primers which hybridize onto the DNA coding for HC110-R according to the invention, or strands complementary thereto, or onto the 5′- or 3′-flanking regions thereof,
b) bringing the oligonucleotide probe or the primers into contact with an appropriately prepared DNA-containing sample,
c) detecting the hybridization of the oligonucleotide or primer (e.g. using the polymerase chain reaction),
d) sequencing the detected sequence of the HC110-R gene, and
e) comparing the sequence with the DNA sequences according to the invention, preferably with the sequence ID No. 2 described in the application DE-A-197 04 024.

The invention therefore likewise relates to a diagnostic test kit for detecting helminths, preferably from the phylum Nematoda, particularly preferably from the family Trichostrongylidae, very particularly preferably of the genus Haemonchus and most preferably of the species Haemonchus contortus, which kit makes available inter alia oligonucleotides as described above which can be used in methods for detecting species from the systematic groups mentioned.

The invention likewise relates to a diagnostic test kit as described above, where the oligonucleotides made available in this test kit are provided with a detectable marker. Such detectable markers may include inter alia enzymes, enzyme substrates, coenzymes, enzyme inhibitors, fluorescent markers, chromophores, luminescent markers and radioisotopes.

This invention also relates to the use of the aforementioned HC110-R polypeptides or fragments thereof and of receptor proteins which are 70%, preferably 80%, particularly preferably 90% and very particularly preferably 95% homologous thereto from helminths, preferably from the phylum Nematoda, particularly preferably from the family Trichostrongylidae, very particularly preferably of the genus Haemonchus and most preferably of the species Haemonchus contortus for producing vaccines which comprise at least one HC110-R polypeptide or fragment or a receptor protein thereof which is 70%, preferably 80%, particularly preferably 90% and very particularly preferably 95% homologous thereto. The vaccine is able in this case to elicit an immune response which is specific for an HC110-R protein described above.

In a preferred embodiment, the vaccine comprises an antigenic determinant, e.g. a single determinant of a polypeptide with an amino acid sequence according to the sequence ID No. 2 described in the application DE-A-197 04 024, or of a polypeptide encoded by the aforementioned DNA or fragments thereof.

The present invention further relates to methods for producing the polypeptides to be used according to the invention. The polypeptides encoded by the known nucleic acids can be produced by cultivating host cells which comprise these nucleic acids under suitable conditions. The desired polypeptides can then be isolated from the cells or the culture medium in a conventional way. The polypeptides can also be produced in in vitro systems.

A rapid method for isolating the polypeptides according to the invention which are synthesized by host cells using a nucleic acid according to the invention starts for example, as described in the examples, with the expression of a fusion protein, where the fusion partner can be affinity-purified in a simple manner. The fusion partner may be, for example, glutathione S-transferase. The fusion protein can then be purified on a glutathione affinity column. The fusion partner can be removed by partial proteolytic cleavage, for example at linkers between the fusion partner and the polypeptide according to the invention which is to be purified. The linker can be designed so that it includes target amino acids, such as arginine and lysine residues, which define sites for cleavage by trypsin. Such linkers can be generated by employing standard cloning methods using oligonucleotides. Another possible method is based on the use of histidine fusion proteins and purification thereof on Ni²⁺ Talon columns.

Further possible purification methods are based on preparative electrophoresis, FPLC, HPLC (e.g. using gel filtration, reverse phase or slightly hydrophobic columns), gel filtration, differential precipitation, ion exchange chromatography and affinity chromatography.

Since receptor-like protein kinases are membrane proteins, detergent extractions are preferably carried out in the purification methods, for example using detergents which affect the secondary and tertiary structures of the polypeptides only slightly or not at all, such as nonionic detergents.

The purification of the polypeptides to be used according to the invention may include the isolation of membranes starting from host cells which express the nucleic acids according to the invention. Such cells preferably express the polypeptides in a copy number sufficient for the amount of the polypeptides found in a membrane fraction to be at least 10 times higher than that found in comparable membranes from cells which naturally express the HC110-R gene; the amount is particularly preferably at least 100 times, very particularly preferably at least 1000 times higher.

The terms “isolation or purification” as used herein mean that the polypeptides are separated from other proteins or other macromolecules of the cell or the tissue. A composition according to the invention containing the polypeptides is preferably enriched at least 10-fold and particularly preferably at least 100-fold, in terms of the protein content, compared with a preparation from the host cells.

The polypeptides according to the invention can also be affinity-purified without fusion partners with the aid of antibodies which bind to the polypeptides.

The invention further relates to antibodies which bind specifically to the aforementioned polypeptides or receptors. Such antibodies are produced in a conventional way. For example, such antibodies can be produced by injecting a substantially immunocompetent host with an amount which is effective for antibody production of a transmembrane receptor according to the invention, such as the HC110-R receptors from H. contortus or of a fragment thereof and by subsequent isolation of this antibody. It is additionally possible to obtain in a manner known per se an immortalized cell line which produces monoclonal antibodies. The antibodies may, where appropriate, be labelled with a detection reagent. Preferred examples of such a detection reagent are enzymes, radiolabelled elements, fluorescent chemicals or biotin. In place of the complete antibody it is also possible to employ fragments which have the desired specific binding properties.

The term “agonist” as used herein refers to a molecule which activates transmembrane receptors.

The term “antagonist” as used herein refers to a molecule which displaces an agonist from its binding site or inhibits the function of the agonist.

The term “modulator” as used herein is generic for agonist and antagonist.

Modulators may be small organic chemical molecules, peptides or antibodies which bind to the polypeptides according to the invention. Modulators may furthermore be small organic chemical molecules, peptides or antibodies which bind to a molecule which in turn binds to the polypeptides according to the invention and thus affects their biological activity. Modulators may be mimetics of natural substrates and ligands.

The modulators are preferably small organic chemical compounds.

The binding of the modulators to the polypeptides may alter the cellular processes in a way leading to the death of the helminths or arthropods treated therewith.

The present invention therefore also extends to the use of modulators of the polypeptides as anthelmintics and arthropodicides.

This invention likewise relates in particular to the use of α-LTX as anthelmintic.

The use according to the invention of the nucleic acids or polypeptides in a method according to the invention also makes it possible to find compounds which bind to the receptors according to the invention. The latter may likewise be employed as anthelmintics, e.g. as nematicides for plants or as anthelmintic active substance for animals. For example, host cells which contain the nucleic acids and express the corresponding receptors or polypeptides, or the gene products themselves, are brought into contact with a compound or a mixture of compounds under conditions which allow interaction of at least one compound with the host cells, the receptors or the individual polypeptides.

The present invention relates in particular to a method which is suitable for the identification of nematicidal active substances which bind to transmembrane receptors from helminths or arthropods, preferably to GPCR of the sekretin subfamily, particularly preferably to the receptor HC110-R from H. contortus, and bind to receptors which are 70%, preferably 80%, particularly preferably 90% and very particularly preferably 95% identical in sequence thereto. The methods may, however, also be carried out with an HC110-R-homologous receptor from a species other than the species mentioned here. Methods which use receptors other than the HC110-R according to the invention are fully encompassed by the present invention.

The methods according to the invention include high throughput screening (e.g. high throughput screening (HTS) and ultra high throughput screening (UHTS)). It is possible to use for this purpose both host cells and cell-free preparations which contain the nucleic acids according to the invention host the polypeptides according to the invention.

Cell-Free Test Systems

Many test systems which aim to test compounds and natural extracts are designed for high throughputs in order to maximize the number of substances investigated in a given period. Test systems which are based on cell-free operations require purified or semipurified protein. They are suitable for an “initial” testing which aims primarily to detect a possible effect of a substance on the target protein.

Effects such as cytotoxicity are usually ignored in these in vitro systems. The test systems moreover examine both inhibitory or suppressive effects of the substances and stimulatory effects. The efficacy of a substance can be checked by concentration-dependent test series. Control mixtures without test substances can be used for assessing the effects.

Cell-Based Test Systems

The development of cell-based test systems for identifying substances which modulate the activity of HC110-R and homologous receptors is made possible by the cell lines stably transformed with HC110-R which are made available by the present invention, but also by the corresponding homologous receptors from other species which can be identified on the basis of the present invention.

The present invention likewise makes it possible to identify other compounds which are effective as calcium channel blockers.

Modulators can be found by incubating a synthetic reaction mix (e.g. products of in vitro translation) or a cellular constituent, such as a membrane or any other preparation which contains the polypeptide, together with a labelled substrate or ligand of the polypeptides in the presence and absence of a candidate molecule which may be an agonist or antagonist. The ability of the candidate molecule to increase or inhibit the activity of the polypeptides according to the invention becomes evident from an increased or reduced binding of the labelled ligand or from an increased or reduced conversion of the labelled substrate. Molecules which bind well and lead to an increased activity of the polypeptides according to the invention are agonists.

Molecules which bind well but do not induce the biological activity of the polypeptides according to the invention are probably good antagonists.

Scintillation Proximity Assay (SPA)

One possibility for identifying substances which modulate the activity of HC110-R and receptor proteins homologous thereto is the so-called scintillation proximity assay (SPA), see EP-A-015 473. This test system makes use of the interaction of a receptor (e.g. HC110-R) with a radiolabelled ligand (e.g. a small organic molecule or a second radiolabelled protein molecule). The receptor is in this case bound to microspheres or beads which are provided with scintillating molecules. During the decay of radioactivity, the scintillating substance in the sphere is excited by the subatomic particles of the radioactive marker and emits a detectable photon. The test conditions are optimized so that the only particles emitted by the ligand which lead to a signal are those emitted by a ligand bound to the receptor or HC110-R.

In one possible embodiment, HC110-R is bound to the beads, either with or without interacting or binding test substances. It would also be possible to employ in this case fragments of the HC110-R receptor. A radiolabelled ligand might be, for example, labelled (α-LTX, nifedipine or a labelled depsipeptide. When a binding ligand binds to the immobilized HC110-R receptor, this ligand would have to inhibit or abolish an existing interaction between the immobilized HC110-R and the labelled ligand in order itself to bind in the region of the contact area. Successful binding to the immobilized HC110-R receptor can then be detected by means of a flash of light.

Correspondingly, an existing complex between an immobilized and a free, labelled ligand is destroyed by the binding of a test substance, which leads to a fall in the detected intensity of light flashes. The test system then corresponds to a complementary inhibition system.

Two-Hybrid System

Another example of a test system based on whole cells is the so-called two-hybrid system. A specific example thereof is the so-called interaction trap. This involves genetic selection of interacting proteins in yeast (see, for example, Gyuris et al. 1993). The test system is designed to detect and describe the interaction of two proteins through successful interaction leading to a detectable signal.

Such a test system can also be adapted for the testing of large numbers of test substances in a given period.

The system is based on the construction of two vectors, the “bait” vector and the “prey” vector. A gene coding for an HC110-R according to the invention or fragments thereof is cloned into the bait vector and then expressed as fusion protein with the LexA protein, a DNA-binding protein. A second gene coding for an HC110-R interaction partner, for example for an α-LTX, is cloned into the prey vector where it is expressed as fusion protein with the B42 prey protein. Both vectors are present in a Saccharomyces cerevisiae host which contains copies of LexA-binding DNA on the 5′ side of a lacZ or HIS3 reporter gene. If an interaction takes place between the two fusion proteins, transcription of the reporter gene is activated. If the presence of a test substance leads to inhibition or destruction of the interaction, the two fusion proteins are no longer able to interact and the product of the reporter gene is no longer produced.

Displacement Test

Another example of a method with which it is possible to find modulators of the polypeptides according to the invention is a displacement test in which, under conditions suitable for this purpose, the polypeptides according to the invention and a potenial modulator are brought together with a molecule which is known to bind to the polypeptides according to the invention, such as a natural substrate or ligand or a substrate or ligand mimetic. α-LTX is preferably used for this in a manner according to the invention.

A known analytical system, e.g. from Biacore AB, Uppsala, Sweden, can be employed for molecular interaction studies using the complete HC110-R protein or the N- host C-terminal deletion mutants of HC110-R, or else variants of the HC110-R molecule which have been modified by in vitro mutagenesis or other known methods. This may entail on the one hand
(i) coupling the HC110-R protein or fragments thereof by known chemical methods (coupling via amines, thiols, aldehydes) or affinity binding (e.g. streptavidin-biotin, IMAC) to a biochip, or on the other hand
(ii) coupling α-LTX or another modulator, e.g. BAY 44-4400 or other possible ligands as described under (i) to the chip.

Binding of a ligand (HC110-R protein or any modulator, e.g. BAY 44-4400 etc.) present in solution to the immobilized molecules is physically measurable. In the Biacore Instrument, the ligand is immobilized on a sensor chip which has a thin layer of gold. The analyte solution is diffused through a microflow cell on the chip.

Binding of the analyte to the immobilized ligands increases the local concentration on the surface, with the refractive index of the medium near the gold layer gradually increasing. This has an effect on the interaction between free electrons (plasmons) in the metal and photons which are emitted from the instrument. These physical changes are proportional to the mass and number of molecules on the chip, and the ligand-analyte binding is recorded in real time, making it possible to determine the apparent association/dissociation rate (Fivash et al. 1998). The specificity of the binding is validated by competition experiments.

Corresponding measurements are also used to determine the HC110-R protein domains important for the binding of ligands, and for identifying novel, previously undisclosed ligands of HC110-R.

Calcium Imaging

Calcium imaging or signalling is to be regarded as another method for detecting substances interacting with HC110-R (see, for example, FIGS. 10 and 11). This entails the use of calcium indicators with whose aid changes in the intracellular calcium level are made detectable. HC110-R-expressing cells which are loaded with calcium indicators are employed in this method. When there is a calcium influx caused by an HC110-R agonist, or when there is release of intracellular calcium, under UV excitation there is a change in the absorption as a function of the calcium loading of the indicator. An antagonist can be identified in such a system by the complete or partial suppression of the calcium signal induced by the agonist (e.g. α-LTX). Possible calcium indicators which are suitable for this purpose are Fura-2 (Sigma) or Indo-1 (molecular probes).

Further calcium indicators can be excited by visible light and vary in their fluorescence characteristics in a detectable manner depending on their calcium loading. The indicators Fluo-3 and Fluo-4 have a high calcium affinity. Fluo-4 is suitable, with its stronger fluorescence signal, in particular for measurements in test systems in which the cells are employed only in low density, as in the case of HEK293 cells. Further indicators are Rhod-2, x-Rhod-1, Fluo-5N, Fluo-5F, Mag-Fluo-4, Rhod-5F, Rhod-5N, Y-Rhod-5N, Mag-Rhod-2, Mag-X-Rhod-1, Calcium Green-1 and -2, Calcium Green-5N, Oregon Green 488 BAPTA-1, Oregon Green 488 BAPTA-2 and −5N, Fura Red, calcein and the like.

An alternative to loading cells with calcium indicators is recombinant expression of photoproteins in the target cells. These photoproteins then respond in the form of light emission after they have formed a complex with calcium ions. A photoprotein which has already been used many times in numerous investigations and test systems is aequorin. The cells expressing the target protein and simultaneously aequorin are first loaded with the luminophor coelenterazine in this test method. The apoaequorin formed by the cells forms a complex with the coelenterazine and carbon dioxide. If calcium subsequently also enters the cells and binds to the complex there is release of carbon dioxide and blue light (emissions maximum ˜466 nm). The light emission in this case correlates with the calcium concentration prevailing inside the cell.

This invention thus likewise relates to the use of HC110-R, fragments thereof or similar proteins from other organisms in a test method in which the function of the receptor or the binding of modulators to the receptor is detected by means of a light signal mediated by aequorin or similar photoproteins.

It is also possible in this way by using host cells or transgenic invertebrates which comprise the nucleic acid according to the invention to find substances which alter the expression of the receptors. Such substances may also represent valuable anthelmintics.

The active substances found with the aid of the method according to the invention are correspondingly suitable for controlling animal pests, in particular insects, arachnids and nematodes which occur in agriculture, in forests, in the storage and protection of materials, and in the hygiene sector. The active substances found by the method according to the invention are particularly suitable for controlling nematodes and arachnids. The abovementioned pests include:
From the order of Isopoda e.g. Oniscus asellus, Annadillidium vulgare, Porcellio scaber.
From the order of Diplopoda e.g. Blaniulus guttulatus.
From the order of Chilopoda e.g. Geophilus carpophagus, Scutigera spp.
From the order of Symphyla e.g. Scutigerella immaculata.
From the order of Thysanura e.g. Lepisma saccharina.
From the order of Collembola e.g. Onychiurus ammatus.
From the order of Orthoptera e.g. Acheta domesticus, Gryllotalpa spp., Locusta migratoria migratorioides, Melanoplus spp., Schistocerca gregaria.
From the order of Blattaria e.g. Blatta orientalis, Periplaneta americana, Leucophaea maderae, Blattella gernanica.
From the order of Dermaptera e.g. Forficula auricularia.
From the order of Isoptera e.g. Reticulitermes spp.
From the order of Phthiraptera e.g. Pediculus humanus corporis, Haematopinus spp., Linognathus spp., Trichodectes spp., Damalinia spp.
From the order of Thysanoptera e.g. Hercinothrips femoralis, Thrips tabaci, Thrips palmi, Franklinella accidentalis.
From the order of Heteroptera e.g. Eurygaster spp., Dysdercus intermedius, Piesma quadrata, Cimex lectularius, Rhodnius prolixus, Triatoma spp.
From the order of Homoptera e.g. Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp.
From the order of Lepidoptera e.g. Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae.
From the order of Coleoptera e.g. Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinaniensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica, Lissorhoptrus oryzophilus.
From the order of Hymenoptera e.g. Diprion spp., Hoplocampa spp., Lasius spp., Monomorium pharaonis, Vespa spp.
From the order of Diptera e.g. Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp., Liriomyza spp.
From the order of Siphonaptera e.g. Xenopsylla cheopis, Ceratophyllus spp.
From the class of Arachnida e.g. Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.

The plant-parasitic nematodes include e.g. Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

The active substances found using the method according to the invention are active not only against plant, hygiene and stored product pests, but also in the veterinary medical sector against animal parasites (ectoparasites) such as hard ticks, soft ticks, mange mites, leaf mites, flies (biting and licking), parasitic fly larvae, lice, hair lice, fur lice and fleas. These parasites include:
From the order of Anoplurida e.g. Haematopinus spp., Linognathus spp., Pediculus spp., Phtirus spp., Solenopotes spp.
From the order of Mallophagida and the suborders Amblycerina and Ischnocerina e.g. Trimenopon spp., Menopon spp., Trinoton spp., Bovicola spp., Werneckiella spp., Lepikentron spp., Damalina spp., Trichodectes spp., Felicola spp.
From the order Diptera and the suborders Nematocerina and Brachycerina e.g. Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.
From the order of Siphonapterida e.g. Pulex spp., Ctenocephalides spp., Xenopsylla spp., Ceratophyllus spp.
From the order of Heteropterida e.g. Cimex spp., Triatoma spp., Rhodnius spp., Panstrongylus spp.
From the order of Blattarida e.g. Blatta orientalis, Periplaneta americana, Blattela germanica, Supella spp.
From the subclass of Acaria (Acarida) and the orders of Meta- and Mesostigmata e.g. Argas spp., Ornithodorus spp., Otobius spp., Ixodes spp., Amblyomma spp., Boophilus spp., Dennacentor spp., Haemophysalis spp., Hyalomma spp., Rhipicephalus spp., Dennanyssus spp., Raillietia spp., Pneumonyssus spp., Sternostoma spp., Varroa spp.
From the order of Actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodectes spp., Laminosioptes spp.

The active substances found with the aid of the method of the invention are also suitable for controlling mites, especially house dust mites, e.g. Dermatophagoides pteronyssinus and D. farinae.

The active substances found using the method according to the invention are also suitable for controlling arthropods which infest agricultural productive livestock such as, for example, cattle, sheep, goats, horses, pigs, donkeys, camels, buffaloes, rabbits, chickens, turkeys, ducks, geese, bees, other pets such as, for example, dogs, cats, caged birds and aquarium fish, and so-called experimental animals such as, for example, hamsters, guinea pigs, rats and mice. Control of these arthropods is intended to reduce deaths and decreases in production (of meat, milk, wool, hides, eggs, honey etc.) so that more economic and easier animal husbandry is possible through use of the active substances according to the invention.

Compounds found with the aid of the described methods and polypeptides are likewise valuable for the treatment of animals and humans infected by pathogenic endoparasites of humans or of productive livestock, pets, zoo animals and laboratory and experimental animals.

The compounds can be used at all stages of development of normal, sensitive strains and resistance strains. It is possible by treatment with compositions which contain one or more of these compounds both to prevent economic losses in productive livestock and treat diseases in humans and animals. The following parasites are of particular interest as targets of the active substances found:
Enoplida, e.g. Trichuris spp., Capillaria spp., Trichomosoides spp., Trichinella spp.
Rhabditia, e.g. Micronema spp., Strongyloides spp.
Strongylida, e.g. Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Bunostomum spp., Globocephalus spp., Syngamus spp., Cyathostomum spp., Cylicocyclus spp., Neostrongylus spp., Cystocaulus spp., Pneumostrongylus spp., Spicocaulus spp., Elaphostrongylus spp., Parelaphostrongylus spp., Crenosoma spp., Paracrenosoma spp., Angiostrongylus spp., Aelurostrongylus spp., Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp. Dictyocaulus spp., Muellerius spp., Protostrongylus spp.
Oxyurida, e.g. Oxyuris spp., Enterobius spp., Passalurus spp., Syphacia spp., Aspi-culuris spp., Heterakis spp.
Ascaridia, e.g. Ascaris spp., Toxascaris spp., Toxocara spp., Parascaris spp., Anisa-kis spp., Ascaridia spp.
Spirurida, e.g. Gnathostoma spp., Physaloptera spp., Thelazia spp., Gongylonema spp., Habronema spp., Parabronema spp., Draschia spp., Dracunculus spp.
Filariida, e.g. Stephanofilaria spp., Parafilaria spp., Setaria spp., Loa spp., Dirofilaria spp., Litomosoides spp., Brugia spp., Wuchereria spp., Onchocerca spp.
Gigantorhynchida, e.g. Filicollis spp., Moniliformis spp., Macracanthorhynchus spp., Prosthenorchis spp.
Mastigophora (Flagellata)
Trypanosomatidae, e.g. Trypanosoma b. brucei, T. b. gambiense, T. b. rhodesiense, T. congolense, T. cruzi, T. evansi, T. equinum, T. lewisi, T. percae, T. simiae, T. vivax, Leishmania brasiliensis, L. donovani, L. tropica
Trichomonadidae, e.g. Giardia lambilia, G. canis.
Sarcomastigophora (Rhizopoda), e.g. Entamoeba histolytica
Hartmanellidae, e.g. Acanthamoeba sp., Hartmanella spp.
Apicomplexa (Sporozoa), e.g. Eimeria acervulina, E. adenoides, E. alabahmensis, E. anatis, E. anseris, E. arloingi, E. ashata, E. auburnensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. labbeana, E. leucarti, E. magna, E. maxima, E. media, E. meleagridis. E. meleagrimitis, E. mitis, E. necatrix, E. ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuernii, Globidium spec., Isospora belli, I. canis, I. felis, I. ohioensis, I. rivolta, I. spec., I. suis, Neospora caninum, Cystisospora spec. Cryptosporidium spec.
Toxoplasmadidae, e.g. Toxoplasma gondii
Sarcocystidae, e.g. Sarcocystis bovicanis, S. bovihominis, S. neuvona, S. ovicanis, S. ovifelis, S. spec., S. suihominis
Leucozoide, e.g. Leucozytozoon simondi
Plasmodiidae, e.g. Plasmodium berghei, P. falciparum, P. malariae, P. ovale, P. vivax, P. spec.
Piroplasmea, e.g. Babesia argentina, B. bovis, B. canis, B. spec., Theileria parva, T. spec.
Adeleina, e.g. Hepatozoon canis, H. spec.
   Also of importance are
Myxospora and Microspora, e.g. Glugea spec. and Nosema spec., and Pneumocystis carinii, Ciliophora (Ciliata), e.g. Balantidium coli, Ichthiophthirius spec., Trichondina spec. or Epistylis spec.

The compounds and compositions found are likewise effective for protozoa of insects such as those of the phylum Microsporidia, particularly those of the order Nosema, very particularly those of the species Nosema apis, which are parasites of honey bees.

The present invention therefore also relates to the use of compounds which have been found using a method according to the invention host using the nucleic acids or polypeptides according to the invention for producing a composition for controlling helminths host arthropods.

### EXAMPLES

1. Construction and Screening of the cDNA Library

The construction of the cDNA library, the production of KLH-PF1022A conjugate and antiserum against PF1022A, and the immunoscreening of the cDNA library and the DNA analysis took place as described in WO 98/15625.

2. Isolation of RNA

The total RNA was extracted and isolated from adult Haemonchus contortus nematodes by the GTC/CsCl cushion method (Sambrook et al. 1989) or obtained by a single step by GTC/phenol/chloroform extraction (Chomczynski and Sacchi 1987). Poly (A)⁺ RNA was isolated by chromatography on oligo(dT)cellulose (Aviv and Leder, 1972).

3. Northern Blotting

Glyoxylated total RNA from Haemonchus contortus (20 μg pe lane) were fractionated in an agarose gel (Sambrook et al. 1989; McMaster and Carmichael 1977) and transferred to a Hybond N membrane (Amersham) by the basic capillary transfer method (Chomczynski, 1992). Radiolabelled probes were prepared by randomized labelling of linearized plasmid DNA (HC110-R) using a Megaprime kit (Amersham, Braunschweig, Germany) and 50 μCi of [α-³²P]dCTP (3000 Ci/mmol, ICN, Meckenheim, Germany). The hybridization was carried out in 6×SSC (1×SSC:0.15 M NaCl, 0.015 M Na citrate), 5× Denhardt's reagent (0.1% polyvinylpyrrolidone, 0.1% BSA, 0.1% Ficoll 400), 0.1% SDS and 100 μg/ml herring sperm DNA at 65° C. overnight. The filters were washed with high stringency in 0.1×SSC and 0.1% SDS at 65° C. and exposed (Kodak BioMax MS films at −80° C.), see FIG. 1(A).

4. 5′- and 3′ RACE PCR

The 5′/3′ RACE method was used to isolate the 5′ and 3′ ends missing in the identified cDNA clone.

The 5′ RACE is based on specific amplification of the 5′ end of a gene from mRNA. A sequence-specific primer and AMV reversed transcriptase are used to synthesize the first cDNA strand. A poly (A) tail is attached to the product, so that it is possible to employ an oligo dT anchor primer and a nested sequence-specific primer in the subsequent PCR. It is possible in a second PCR to use another nested primer in order to ensure specifity.

In the 3′ RACE, the first cDNA strand is synthesized using an oligo dT primer and the subsequent PCR reactions take place with sequence-specific primers.

cDNA was synthesized from 1 μg of total H. contortus RNA using the primer 5′-GGT CAC CGT CGT CCC AGA AA-3′ and the 5′ RACE kit from GIBCO BRL (Eggenstein, Germany). The Superscript reverse transcriptase was used for this purpose. C tailing of the C terminus of the cDNA was carried out using terminal deoxynucleotidyltransferase. The first amplification took place with 400 nM of an oligodeoxyinosyl anchor primer (5′-CUA CUA CUA CUA GGC CAC GCG TCG ACT AGT ACG GGI IGG GII GGG IIG-3′) and 400 nM of the first nested primer 5′-CCA TTC GAT TCC TCT TCT CG-3′ (Birsner und Grob, Denzlingen, Germany) in 50 μl containing 200 μM of each dNTP, 1.5 mM MgCl₂, a fifth of the tailed cDNA and 2.5 U of native Taq polymerase. The first denaturation took place at 94° C. for 5 minutes, followed by 35 cycles each of 1 minute at 94° C., 1 minute at 53° C. and 2 minutes at 72° C., in turn followed by a last synthesis step of 10 minutes at 72° C. The reaction conditions for the nested PCR were the same as described above with the exception that 1 μl of the first amplification product was used as template, and the gene-specific second nested primer 5′-GTC GAT GGT GCA GAT TTC GC-3′, a truncated form of the anchor primer (5′-CUA CUA CUA CUA GGC CAC GCG TCG ACT AGT AC-3′) and only 25 cycles were carried out at an annealing temperature of 53° C.

The 3′ RACE PCR (GIBCO BRL, Eggenstein, Germany) was carried out with 1 μg of the total RNA from adult H. contortus and 500 nM of the oligo dT adapter primer 5′-GGC CAC GCG TCG ACT AGT ACT TTT TTT TTT TTT TTT T-3′, starting with a preincubation at 70° C. for 10 minutes and at 4° C. for 2 minutes. After addition of 2.5 mM MgCl₂, 500 μM of each dNTP, 10 μM DTT and 200 U of Super-Script II reverse transcriptase in 20 μl, the cDNA was incubated at 42° C. for 50 minutes and, in a final step, at 70° C. for 15 minutes and at 4° C. for 10 minutes. The RNA was removed by 2 U of E. coli RNase H and incubation at 37° C. for 10 minutes. The first amplification took place with 400 nM of a universal adapter primer (5′-CUA CUA CUA CUA GGC CAC GCG TCG ACT AGT AC-3′) and 400 nM of the sequence-specific primer 5′-TTTGTTCTT CCT TOG TAT CC-3′ in 50 μL containing 200 mM of each dNTP, 1.5 mM MgCl₂, a tenth of the tailed cDNA and 2.5 U of native Taq DNA polymerase. The first denaturation step took place at 94° C. for 3 minutes, followed by 35 cycles each of 1 minute at 94° C., 1 minute at 51° C. and 2 minutes at 72° C., and a final synthesis step of 15 minutes at 72° C. 2 μl of the first amplification were used for the nested PCR, with the conditions otherwise identical, but now using the shorter adapter primer 5′-GGC CAC GCG TCG ACT AGT AC-3′ and the nested primer 5′-ACA CTC TAA TCT CCA ACT G-3′ with 30 cycles in this case. The PCR products were analyzed on 2% agarose gels, eluted and cloned into the TA vector pMosBlue (Amersham, Braunschweig, Germany).

5. Transfer of the RNA

Transfer of the fractionated RNA from the glyoxal gel and of genomic DNA from a TBE agarose gel to a neutral Hybond N nylon membrane [Amersham, Braunschweig] takes place by the downward alkaline capillary transfer in alkaline transfer solution by the method of Chomczynski (1992) for 2 h. The membrane is then neutralized in 0.2 M sodium phosphate buffer (pH 6.8) for 20 min, dried and baked at 80° C. for 20-60 min. For the hybridization with radiolabelled probes, the membrane is sealed with 5 ml/cm² prehybridization solution in plastic bags and incubated at 65° C. for 3 h. The buffer is replaced by hybridization solution for the hybridization. On use of Rapid Hyb. solution [Amersham, Braunschweig], the prehybridization takes place at 65° C. without additional prehybridization solution in 30 min. After addition of the probe radiolabelled by random priming, the hybridization takes place at 65° C. overnight.

The membrane is then washed once with 2×SSC/0.1% SDS at 65° C. for 20 min and three times with 0.1×SSC/0.1% SDS for 1 h each time. The membrane is exposed to Kodak X-OMAT or Kodak BIOMAX-MS X-ray films with appropriate intensifying screen at −80° C.

6. In vitro Transcription and Translation

The TNT T7/T3-coupled reticulocyte lysate system (Promega) was used to transcribe and translate the full length of the coding sequence of HC110-R in the presence of ³⁵S-labelled methionine and cysteine (ICN, Eschwege, Germany) in accordance with the manufacturer's protocols. The RNA was translated using a rabbit reticulocyte lysate system (Promega, Serva, Heidelberg) using a 40 μCi ³⁵S-labelled mixture (>1000 Ci/mmol, ICN, Meckenheim, Germany), 1 μg of circularized HC110-R plasmid DNA and 10 U of T3 RNA polymerase. The reaction was carried out at 30° C. for 90 minutes. A positive control contained 1 μg of luciferase control DNA.

The reaction products were fractionated by SDS polyacrylamide gel electrophoresis (Lämmli 1970) and fluorographed using the amplify fluorography solution (Amersham Pharmacia Biotech) or 1 M sodium salicylate (pH 7) (Chamberlain 1979), and the gels were then dried and exposed (Kodak BioMax MR film with intensifier at −80° C.), see FIG. 1(B).

Alternatively, the MAXIscript In Vitro Transcription Kit from Ambion (Heidelberg) was used for the in vitro synthesis of an RNA transcript from HC110-R cDNA cloned into pBluescript SK. 4 μg of HC110-R plasmid DNA were linearized with the restriction enzyme Hind III or Sal I behind the stop codon, extracted with phenol/chloroform, precipitated in 3 vol. of ethanol and dissolved in DEPC/H₂O. 2 μg of HC110-R cDNA in a 50 μl reaction mixture were mixed with 2.5 μl of 200 mM DTT, 2.5 μl each of 10 mM ATP, CTP, GTP and UTP, 5 μl of 10× transcription buffer, 1.6 μl of RNasin inhibitor (40 μl) (Promega, Heidelberg) and 2 μl of T3 phage polymerase (10 U/μl) and incubated at 37° C. for 2 h. After 1 h, a further 2 μl of T3 phage polymerase (10 U/μl) were added. The mixture was mixed with 1.5 μl of RNase-free DNase I (2 U/μl) and 1 μl of RNasin inhibitor (40 U/μl), incubated at 37° C. for 15 min, extracted with phenol/chloroform, precipitated in 3 vol. of ethanol and resuspended in 25 μl of DEPC/H₂O. ⅕ of the volume of the sample was analyzed in a glyoxal gel.

7. DNA Analysis

The clones were sequenced by the dideoxynucleotide chain termination method with the assistance of automated laser fluorescence sequencing (LICOR 4000; MWG, Ebersberg, Germany), and the Thermo Sequenase fluorescent-labelled cycle sequencing kit (Amersham, Braunschweig, Germany). The sequences of both strands were determined, and the sequence data were [lacuna] with the GCG software (Genetics Computer Group Inc., Madison, Wis., USA) and the PC/GENE software (Intelligenetics, Mountain View, Calif., USA). The programmes FASTA (Pearson and Lipman 1988), BLITZ (Smith and Waterman 1981) and BLAST (Altschul and Lipmann 1990) were used to screen the EMBL and Swiss-Prot protein databases for sequence similarities of the derived protein sequence. The protein sequences were analyzed using SAPS (Brendel et al. 1992) and using PROSITE and Prot-Param (Appel et al. 1994).

8. Preparation of Protein Extracts from Haemonchus contortus

50-100 mg of H. contortus worms frozen in liquid nitrogen were taken up in 1 ml of TRIZOL [Gibco, Karlsruhe]. The nematodes were homogenized together with the TRIZOL in a glass potter for 3×15 sec and incubated at RT for 5 min. After addition of 200 μl of chloroform per ml of TRIZOL and shaking of the sample for 15 sec, the mixture was incubated at RT for a further 2-3 min, before it was centrifuged at 7 000-12 000 rpm and 4° C. for 10 min. The upper aqueous phase contains the RNA, the interphase the genomic DNA and the red organic phase the proteins (Coombs et al. 1990; Chomczynski 1993). The aqueous phase was removed and worked up separately. The interphase and organic phase were mixed with 300 μl of 100% ethanol per ml of TRIZOL, thoroughly mixed and incubated at RT for 2-3 min. After centrifugation at 2 000 rpm and 4° C. for 5 min, the protein supernatant was cautiously transferred into a new Eppendorf vessel and precipitated with 1 ml of isopropanol at RT for 10 min. Centrifugation was again carried out at 12 000 rpm and 4° C. for 10 min, the supernatant was discarded, and the protein pellet was mixed with 2 ml portions of 0.3 M guanidine hydrochloride solution in 95% ethanol, vortexed, incubated at RT for 20 min and centrifuged at 7 500 rpm and 4° C. for 5 min, 3 times.

The pellet was then dissolved in 2 ml of 100% ethanol, precipitated at RT for 20 min and pelleted at 7 500 rpm and 4° C. for 5 min. The pellet was briefly dried in vacuo and resuspended in urea lysis buffer (8 M). Insoluble material was removed by centrifugation at 10 000 rpm and 4° C. for 10 min, and the supernatant was transferred into a new Eppendorf vessel and, after determination of the protein concentration, stored at −20° C. until processed further.

9. Preparation of Protein Extracts from Yeast Cells

Protein extracts were prepared from a yeast culture by inoculating 5 ml of YPAD medium with a single yeast colony and shaking at 30° C. until saturated (2 days). 2 ml portions of such a yeast culture were centrifuged at 3 000 rpm and 4° C. (Heraeus Biofuge 15 R) for 2 min, washed once with H₂O, resuspended in 250 μl of yeast lysis buffer, transferred into a small test tube, charged with glass beads (Ø 0.5 mm) up to just below the liquid level and vortexed at the highest setting for 5 min. Then 4× RotiLoad buffer (Roth, Karlsruhe) was added, and the mixture was incubated at 95° C. for 5 min. The disrupted cells were transferred into an Eppendorf vessel, and the cell detritus was removed by centrifugation at 14 000 rpm (Eppendorf Centrifuge 5415 C) for 5 min. 20 μl portions of the samples prepared in this way were loaded onto an SDS polyacrylamide gel.

10. Preparation of Protein Extracts from E. coli Cells

Protein extracts were prepared by pelleting 1×10⁷ E. coli cells from a 5 ml overnight culture at 5 000 rpm and 4° C. in a Heraeus floor centrifuge for 5 min, washed with 5 ml of PBS and again centrifuged. The pellet was then resuspended in 1 ml of TRIZOL (Gibco, Karlsruhe) and processed further.

As an alternative to this, the PBS-washed cell pellet was resuspended in PBS again and the cells were broken up by means of several short ultrasound pulses (Sonifier B-12, Branson Sonic Power Company, Danbury, U.S.A.), liquid nitrogen and by addition of lysozyme.

Following the protein determination, 4×RotiLoad buffer (Roth, Karlsruhe) was added and the fraction was analyzed by loading onto an SDS polyacrylamide gel. The total amount of protein loaded per lane was 10-20 μg.

11. Preparation of Protein Extracts from Cell Culture

A confluent 35 mm cell culture dish of adherent cells or 5-10×10⁶ non adherent mammalian cells in FCS-containing medium were previously removed by trypsinization with a trypsin/EDTA solution or detached mechanically after exposure to cold using a cell scraper, transferred into an Eppendorf vessel, pelleted at 13 000 rpm and RT for 10 sec, washed twice with PBS and again centrifuged. After the cells had been taken up in 1 ml of TRIZOL they were lysed by vigorous vortexing or by drawing the cells several times through the needle of a disposable syringe, and incubated at RT for 5 min. As an alternative to this it is also possible for the cell pellet to be resuspended in 1 ml of PBS or urea lysis buffer (8 M) and subjected to a very brief ultrasound treatment (Sonifier B-12, Branson Sonic Power Company, Danbury, U.S.A.). The protein content is then determined by the method of Bradford (1976) or Lowry et al. (1951).

12. Inducible Protein Expression in E. coli

Polyclonal antibodies against HC110-R protein were produced by three HC110-R fragments—the complete HC110-R cDNA, the N-terminal end without TM domains and the C-terminal end after the 7th TM domain—being cloned into the expression vector pRSET B (Invitrogen, Leek, NL) and thus fused N-terminally to a 6×His tag while complying with the reading frame.

The complete coding region of HC110-R was amplified using the P84_ATG BamHI 5′ primer 5′-CTG CCG GAT CCT CGG TTT AAT ACC AAC ATG AGG-3′ and the P3121_TGA HindIII 3′ primer 5′-GCA CTA AGC TTG ACT GAA GCG CAC AAC CTC G-3′, the N terminus up to the 1st transmembrane domain was amplified using the P84_ATG BamHI 5′ primer (see above) and the P1434_TGA HindIII 3′ primer 5′-GGC TCA AGC TTA TCA GAG AAC AAG CGA CAC GGC-3′, and the C terminus starting after the 7th transmembrane domain was amplified using the P2486_ATG BamHI 5′ primer 5′-CTA TCG GAT CCC AAC ATG GCT GGC TCC CGT GAT ACC TCT AGG-3′ and the P3121_TGA HindIII 3′ primer (see above). The annealing temperature for all three plasmid PCRs was initially 56° C. for 5 cycles and then 62° C. for 30 cycles. The PCR products were digested with the enzymes BamHI and HindIII and ligated in a directed manner into the pRSET B expression vector which had likewise been linearized with BamHI/HindIII.

In the pRSET B vector, expression is controlled by a viral promoter of bacteriophages T7. The cloning therefore took place in the XL1-Blue E. coli strain which contains no T7 RNA polymerase gene. The recombinant plasmid was then transformed into T7 polymerase-expressing BL21(De3)pLysS E. coli cells which additionally contain the plasmid pACYC184 which is stabilized by a chloramphenicol-resistence, and which express small amounts of the T7 lysozyme, a natural inhibitor of T7 RNA polymerase. Since these cells are under the control of the lacUV5 promoter, IPTG induction leads to expression of the T7 RNA polymerase and thus also to that of the fusion protein.

A single colony with the required HC110-R plasmid was transferred from a fresh LB plate with 50 Lg/ml ampicillin and 35 μg/ml chloramphenicol into 50 ml of LB medium under the same selection pressure and shaken at 280 rpm and 37° C. overnight up to the stationary phase. The overnight culture was then diluted to OD₆₀₀=0.3, and 100 ml of the culture were shaken further at 280 rpm and 37° C. until OD₆₀₀=0.6-0.5.

1 OD₆₀₀ unit was removed, and the uninduced cells were briefly centrifuged and taken up in 150 μl of 8 M urea lysis buffer, pH 8.0, and 50 μl of 4×RotiLoad buffer [Roth, Karlsruhe]. The uninduced sample was denatured for 2 min, the genomic DNA was sheared by short ultrasound pulses of a few seconds in a Sonifier B-12 [Branson Sonic Power Company, Danbury, U.S.A.], and insoluble particles were pelleted by centrifugation at 14 000 rpm for 3 minutes.

Expression of the fusion protein was induced by adding 1 mM IPTG, and the 100 ml of culture were incubated at 37° C. for a further 3-4 h. After the incubation, the induced cells were centrifuged at 5 000 rpm and 4° C. (Heraeus fluorocentrifuge) for 15 min, and the pellet was washed in PBS and then resuspended in 8 ml of 8 M urea lysis buffer. The cells were disrupted by immersing the cells in liquid nitrogen and then storing at 37° C. 3 times. After the first nitrogen treatment, 0.75 mg/ml lysozyme was added. The last incubation in liquid nitrogen was followed by incubation at 16° C. for 20 min and then by short ultrasound pulses each of 10 sec while cooling in an ice-water bath until the solution had a water-like viscosity. After centrifugation at 13 000 rpm and 4° C. (Beckman J2-21; JS 13.1-Rotor) for 10 minutes, an induced 150 μl sample was removed and mixed with 50 μL of 4×RotiLoad buffer (Roth), and the induction of the particular HC110-R protein fragment together with the uninduced sample was checked by SDS-PAGE with subsequent Coomassie staining or Western blot analysis with a mouse anti-His antibody.

The remaining supernatant was transferred into a fresh vessel and stored at −20° C. for further purification by affinity chromatography.

13. Protein Purification by Metal Affinity Chromatography

The enrichment took place under denaturing conditions via the N-terminal 6×His tag using the IMAC systems (‘Immobilized Metal Affinity Chromatography’) on TALONspin columns from Clontech [Palo Alto, U.S.A.]. The resin in the column was first separately equilibrated with 5 volumes of 8 M urea lysis buffer, pH 8.0, sedimented at 3 000 rpm and 4° C. for 4 min and incubated together with the HC110-R protein supernatant at RT with gentle shaking for 20 min. After centrifugation again, the resin was washed 3 times with 10 times the volume of 8 M urea lysis buffer, pH 8.0, with gentle shaking at RT for 10 min, and again centrifuged. After the last washing step, the pellet was taken up in 1 ml of 8 M urea lysis buffer and used to load the column; the latter was subsequently washed twice with 3 times the volume of 8 M urea lysis buffer before elution with imidazole-containing 8 M urea lysis buffer in several fractions each of 150 μl. The content of fusion protein in the fractions is measured by gel analysis and Bradford protein determination.

14. Protein Purification by Affinity Chromatography

Protein purification by metal affinity chromatography is to be described by way of example here. The enrichment took place under denaturing conditions via the N-terminal 6×His tag using the IMAC systems (‘Immobilized Metal Affinity Chromatography’) on TALONspin columns from Clontech [Palo Alto, U.S.A.]. The resin in the column was first separately equilibrated with 5 volumes of 8 M urea lysis buffer, pH 8.0, sedimented at 3 000 rpm and 4° C. for 4 min and incubated together with the HC-1 protein supernatant at RT with gentle shaking for 20 min. After centrifugation again, the resin was washed 3 times with 10 times the volume of 8 M urea lysis buffer, pH 8.0, with gentle shaking at RT for 10 min, and again centrifuged. After the last washing step, the pellet was taken up in 1 ml of 8 M urea lysis buffer and used to load the column; the latter was subsequently washed twice with 3 times the volume of 8 M urea lysis buffer before elution with imidazole-containing 8 M urea lysis buffer in several fractions each of 150 μl. The content of fusion protein in the fractions is measured by gel analysis and Bradford protein determination.

15. Amino Acid Sequence Analysis

To check the deduced amino acid sequence starting from the cDNA full-length clone HC110-R, the 3′ end consisting of 688 bp (Pos. 2486-3182) with a preceding start codon and Kozak sequence was cloned into the pRSET B-expression vector, and expression of the 21 kD protein (189 AA) in competent BL21(DE3)pLysS E. coli cells was induced by adding 1 mM IPTG. The fusion protein provided with an N-terminal His tag was enriched on a Talon matrix and concentrated and desalted by means of Centricon tubes. Because of the N-terminal His tag, a partial C-terminal protein sequencing of 1 nM of the 21 kD HC110-R protein was carried out by stepwise Schlack-Kumpf degradation (Schlack et al. 1926) in a modification of Boyd (Boyd et al. 1992) by TopLab (Martinsried). Sequencing of the eliminated amino acids took place in a PROCISE 492 amino acid sequence (PE Applied Biosystems, Weiterstadt) and was analyzed using the PROCISE C reversed phase HPLC system consisting of an ABI 140 C microgradient system and an ABI 785A UV/VIS detector and identified using the PROCISE C control software and the ABI data analysis software.

25 pmol of the 21 kD HC110-R protein were cleaved internally with 2% trypsin (Roche Molecular Biochemicals, Mannheim) at 37° C. overnight. A total of four peptide fragments was selected by comparison of the HC110-R standard chromatogram after trypsin digestion with the chromatogram of the trypsin blank digestion in order to preclude sequencing of tryptic autolysis products. These four peptide fragments were subjected to partial N-terminal protein sequencing by the method of stepwise Edman degradation in a modification by Hunkapiller et al. (1983) by TopLab (Martinsried). The cleaved peptide fragments were separated and fractionated by HPLC. The peptide fraction blotted onto Immobilon was introduced into the reaction chamber of the PROCISE 492 amino acid sequencer (Applied Biosystems, Weiterstadt) and the amino acids were separated in a 140 C-PTH-analyzer and UV detector 785 A (Applied Biosystems, Weiterstadt). The amino acids were quantitated by reversed phase HPLC and identified by comparison of retention times with a standard chromatogram constructed before the sequence analysis.

16. Cell Culture Lines

The following cell culture lines were purchased from the Deutsche Sammlung von Microorganismen und Zellkulturen GmbH [Braunschweig] [Drexler et al. 1995]:
COS-7 cells (DSM: ACC 60)—monkey, kidney
HEK-293 cells (ATCC: CRL 1573)—human, embryonic, kidney
   17. Cultivation of the Various Cell Culture Lines

The adherent cell lines COS-7 and HEK-293 were cultivated in 110 mm tissue culture dishes [Greiner, Solingen] in a volume of 10 ml of medium at 37° C., 5% CO₂ and 95% humidity. The cell culture was maintained by cultivating COS-7- and HEK-293 cells in DMEM medium. The media contained 3.024 g/l NaHCO₃, 10% FCS, 50 U/ml penicillin and 50 μg/ml streptomycin and were heated to 37° C. before use. For subcultivation of the COS-7 cells, they were stored at 4° C. for 2 h and then detached mechanically from the culture dish using a cell scraper. The HEK-293 cells could be detached directly from the bottom of the culture dish using a glass pipette.

18. Transient and Stable Transfection of Eukaryotic Cells

The non-liposomal transfection reagent FuGENE 6 from Roche Molecular Biochemicals (Mannheim) was employed for transient introduction of foreign DNA into mammalian cells (Kurachi et al. 1998). For this purpose, about 0.5-1.5×10⁵ cells in 2 ml of medium were put on 35 mm tissue culture dishes in which a sterile glass slide coated with 1% gelatin was placed for subsequent confocal laser scanning microscopy. The cells were cultivated at 37° C., 5% CO₂ and 95% humidity overnight. The medium was changed again before the transfection. For the transfection, 3 μl of FuGENE 6 [Roche Molecular Biochemicals] were diluted in 97 μl of serum-free medium and incubated at RT for 5 min for each reaction mixture. 100 μl portions of the diluted FuGENE 6 were pipetted dropwise onto 1-2 μg of plasmid-DNA (0.5-1 μg/μl) for each mixture and, after cautious mixing, incubated at RT for a further 15 min. The complete reaction mixture was then added dropwise to the cells, and the transfection mixture was distributed uniformly by gently swirling the dish. The cells were cultivated further for 1-2 days without changing the medium. The following 3 negative controls were always included with each transfection: a 35 mm tissue culture dish with 0.5-1.5×10⁵ cells was not transfected, DNA, but no FuGENE 6, was added to the transfection mixture in the second dish, and FuGENE 6, but no DNA, was added to a third dish.

For stable transfection, the desired plasmid HC110-R was cloned in the correct reading frame into the slightly modified expression vector pSecTag A [Invitrogen, Leek, NL] or pIRESneo [Clontech, Palo Alto, U.S.A.]. These vectors harbour a resistance gene as marker so that on addition respectively of Zeocin and G418 or bleomycin 48 72 h after the transient transfection and at every subsequent change of medium only successfully transfected cells which permanently express the resistance gene product survive. The optimal concentration of Zeocin- or G418-containing selection medium was determined beforehand by setting up serial dilutions of the respective cell line.

19. Cellular Localization of Recombinant Proteins in Mammalian Cells

The complete coding region of the HC110-R DNA was cloned into the HindIII/SalI sitee of pEGFP-N3 in order to link the HC110-R protein C-terminally to GFP (green fluorescent protein). The 137 kDa fusion protein is expressed in transiently transformed recipient cell lines, which can be demonstrated by Western blot analysis. The CLSM (confocal laser scanning microscopy) shows that the HC110-R-GFP fusion protein is localized in the cytoplasm of COS-7 and HEK-293 cells and, to a smaller extent, also on the plasma membrane (see also FIG. 8).

A Zeiss IM 35 microscope (Zeiss, Oberkochen) with a Leica CLSM attachment TCS NT (‘Confocal Laser Scanning Microscope Unit’, Leica Lasertechnik, Heidelberg), version 1.5.451, was used for the confocal laser scanning microscopy. Fluorescence of the GFP protein and of the dye fluorescein isothiocyanate (FITC) was excited at 488 nm with an argon laser and that of rhodamine, Texas red, phycoerythrin, Alexa 568, LysoTracker™ Red DND-99, MitoTracker™ Red CMX Ros and propidium iodide was excited at 568 nm with a krypton laser. Z-Series of optical sections through the cell were scanned with a resolution of 1024×1024 pixels and a thickness of 0.5 μm (Giese et al. 1995). Evaluation took place with the AVS software (Advanced Visual Systems Inc., Waltham, Mass., U.S.A.) and later with Adobe Photoshop 5.0 and Corel Draw 8.0 for Windows.

The fusion protein is located in particular in acidic lysosomes, as shown by colocalization using Lysotracker™, a probe for labelling acidic organelles. The vesicles containing the HC110-R protein were observed in increased numbers near the nucleus. A fusion protein of GFP and the mouse β₂-adrenergic GPCR was prepared as control (Accession Number X155643, Nakada et al. 1989), and transfection thereof resulted in the same distribution pattern within the cell as in the case of the HC110-R-GFP fusion protein (FIG. 8).

20. EGFP Constructs for Transient Expression

The vectors pEGFP C1 and pEGFP N3 (Clontech, Palo Alto, Calif., U.S.A.) were cut with the restriction enzymes Hind III and Sal I. Amplification of the HC110-R full-length cDNA took place with the P83EGFP_ATG HindIII 5′ primer 5′-GGT AGA AGC TTT TCG GTT TAA TAC CAA CAT GAG G-3′ and the P3057EGFP_o.TGA SalI 3′ primer 5′-CTG TGT CGA CAA CAT TTC GCC AAT AGT TAG G-3′ at an annealing temperature of 65° C. The PCR product was then likewise cut with the enzymes Hind III and SalI, and was ligated to generate an open reading frame between the Hind III and SalI cleavage sites of the particular vector and was transformed. The resulting fusion protein with the N-terminal GFP tag was called GFP-HC110-R and that with the C-terminal EGFP tag was called HC110-R-GFP. A mouse O₂ adrenergic receptor (GenBank Accession No.: P18762; Nakada et al. 1989) was fused with a C-terminal EGFP tag by amplifying the full-length cDNA with the P117 mouse 2AR XhoI 5′ primer 5′-TAC CTC GAG CTG CTA ACC TGC CAG CCA TG-3′ and the P1349 mouse β₂AR EcoRI 3′ primer 5′-TGT AGA ATT CTT CCT TCC TTG GGA GTC AAC GCT-3′ using an annealing temperature of 55/60° C., cutting with the restriction enzymes XhoI and EcoRI and ligating into the XhoI-EcoRI linearized pEGFP N3. The full-length cDNA of a human muscarinic receptor 1 (huM1Rez.; GenBank Accession No.: Y00508; Allard et al. 1987) which had previously been amplified with the P70HumM1Rez XhoI 5′ primer 5′-ATA TCT CGA GAG CCC CAC CTA GCC ACC ATG AAC A-3′ and the P1465HumM1Rez EcoRI 3′ primer 5′-GAC GAA TTC CAT TGG CGG GAG GGA GTG CGG T-3′ at 55/60° C. was likewise ligated into the XhoI-EcoRI-cut pEGFP N3 vector. The resulting fusion proteins with an open reading frame were called mouseβ₂AR-EGFP and huM1Rez-EGFP.

21. HC110-R-MycHis Tag Constructs for Stable or Transient Transfection

The vector pMyc6×His is derived from the vector pSecTagA [Invitrogen, Leek, NL] by double digestion with the restriction enzymes NhiI and SfiI, followed by blunting of the ends and religation of the vector. The coding region of HCl 0-R was amplified using the PCR primers P83MycTag_ATG BamHI 5′ primer 5′-ATA GGA TCC TTC GGT TTA ATA CCA ACA TGA GG-3′ and P3058MycTag_o.TGA XbaI 3′ primer 5′-CCT GTC TAG AAA CAT TTC GCC AAT AGT TAG G-3′ at an annealing temperature of 56/60° C., cut with the enzymes BamHI and XbaI, and ligated into the pMyc6×His vector which had likewise been linearized with BamHi XbaI and transformed into E. coli DH5α cells. Subsequently, COS-7 cells were stably transfected with the construct and maintained under Zeocin selection pressure.

In parallel with this, the HC110-RMycHis cDNA was amplified with the primers P83_ATGNotI-5′ 5′-ATA TTG CGG CCG CTT CGG TTT AAT ACC AAC ATG-3′ and pMycHis_TGABamHI-3′ 5′-CGC GGA TCC TAG AAG GCA. CAG TCG AGG-3′, then cut and ligated into the bicistronic expression vector pIRES1neo (GenBank Accession No.: U89673) (Clontech, Palo Alto, U.S.A.) which had likewise been restricted with BamHI and NotI. The pIRES1neo vector additionally contains an internal ribosome binding site (IRES) of the encephalomyocarditis virus (ECMV) shortly before the start ATG of the neomycin resistance gene (Rees et al. 1996). In this way, two open reading frames, that of HC110-R and that of the antibiotic resistance marker, are translated from only one mRNA with a human cytomegalovirus (CMV) promoter (Jackson et al. 1990; Jang et al. 1988). Selection took place with G418 (Calbiochem-Novabiochem, La Jolla, Calif., U.S.A.) in COS-7 and HEK-293 cells.

22. Confocal Laser Scanning Microscopy

A Zeiss IM 35 microscope (Zeiss, Oberkochen) with a Leica CLSM attachment TCS NT (‘Confocal Laser Scanning Microscope Unit’, Leica Lasertechnik, Heidelberg), version 1.5.451, was used for the confocal laser scanning microscopy.

Fluorescence was excited at 488 nm with an argon laser and at 568 nm with a krypton laser. Z-Series of optical sections through the cell were scanned with a resolution of 1024×1024 pixels and a thickness of 0.5 μm (Giese et al. 1995). Evaluation took place with the AVS software (Advanced Visual Systems Inc., Waltham, Mass., U.S.A.) and later with Adobe Photoshop 5.0 and Corel Draw 8.0 for Windows.

23. Studies of Binding of α-LTX to HC110-R Fragments

Proteins (20 μg/lane) were fractionated by SDS-PAGE (Lämmli et al., 1970) and electroblotted onto nitrocellulose membranes. In the case of α-LTX binding experiments, the blots were incubated with α-LTX at a concentration of 20 nM in TST at room temperature for 2 h. After washing with TST three times, an anti-α-LTX antibody (from rabbits, Calbiochem-Novabiochem) was used at a concentration of 0.1 μg/ml and an antibody conjugated with peroxidase and directed against rabbit IgG (from goat, dilution: 1:25 000) was used. The antibodies were detected in all experiments using ECL (Amersham-Pharmacia). See also FIG. 9.

24. Calcium Imaging

The intracellular free Ca²⁺ concentration [Ca²⁺]ᵢ was measured in COS-7 and HEK293 cells transiently transfected with the HC110-R-EGFP or β₂-AR-EGFP construct using the calcium imaging method (see also FIG. 11). In each case 2×10⁵ COS-7 and HEK293 cells were applied to a 42 mm cover glass coated with 1% gelatine in 55 mm tissue culture dishes with 5 ml of DMEM medium (with 10% FCS and pen/strep). The transfection took place with the aid of the non-liposomal transfection reagent FuGENE 6 (Roche Molecular Biochemicals, Mannheim). This entailed 6 μl of FuGENE 6 being incubated in 200 μl of DMEM medium without FCS initially at RT for 5 min and, after addition of 4 μg of plasmid DNA, at RT for a further 15 min, before the complete transfection mixture was pipetted dropwise onto the cells. Constructs of the HC110-R full-length clone with a GFP tag fused in the correct reading frame, and the complete cDNA of the β₂-adrenergic receptor which had likewise been provided with a GFP tag at the 3′ end, were transfected. 4 μg of plasmid DNA of the pure pEGFP-N3 expression vector (Clontech, Heidelberg) and untransfected cells to which the transfection reagent FuGENE 6 had been added without DNA served as controls.

Loading of the cells with 1 μM Fura-2/acetoxymethyl ester (Fura-2/AM) (Sigma, Deisenhofen) took place 48 h after the transfection in an Na⁺-HBS solution (150 mM NaCl; 5.4 mM KCl; 1.8 mM CaCl₂; 0.8 mM MgSO₄ 7H₂O; 20 mM glucose; 20 mM Hepes in H₂O, pH 7.3) at 37° C., 5% CO₂ and 95% humidity for 30 min. After the incubation, the cells loaded with Fura-2/AM were examined under an inverted microscope (Zeiss, Axiovert, Germany) and, in parallel with this, in a digital imaging fluorescence microscope (PTI) and differentiated into transfected and untransfected cells at a wavelength of 440 and 490 nm respectively. On average, 5-7 transfected and non transfected cells were selected by fixing an ROI (region of interest). The extinction was measured at 340 nm (calcium-bound Fura-2/AM) and 380 nm (free Fura-2/AM), and the emission was measured at 510 nm. Evaluation took place using the Image Master 1.x software by forming the quotient of 340:510 nm, 380:510 nm and the ratio of 340/380:510 nm (background corrected images) as a function of the agent added.

Agents were always added 6 min after starting the measurement by removing an appropriate volume of Na⁺ HBS buffer and pipetting the agent directly into the sample vessel for a further 30 to 50 min. The total volume in the sample vessel was constant at 1.5 ml throughout the experiment. Initially 30 nM and 75 nM α-latrotoxin (α-LTX, RBI. Natick, U.S.A.) were added to the COS-7 cells expressing the HC110-R-GFP fusion protein, and various concentrations of α-LTX (7.5 nM; 25 nM; 50 nM, 75 nM, 90 nM and 120 nM), to determine the dose-dependency, and various concentrations of the cyclic depsipeptide BAY 44-4400 (100 ng/ml (89.3 nM), 333 ng/ml (297 nM); 400 ng/ml (357 nM), 1 μg/ml (893 nM), 10 μg/ml (8.9 μM), to determine the optimal active concentration, were added to the HEK-293 cells expressing HC110-R-GFP, in each case 6 min before starting the experiment. In some experiments, the HEK-293 cells were preincubated with 4 or 400 ng/ml BAY 44-4400, or with 4 or 400 ng/ml of the optical antipode PF1022-001, which has no anthelmintic activity, in Na⁺ HBS at 37° C., 5% CO₂ and 97% humidity for 90 min, and the cells were loaded after 60 mm by adding 1 μM Fura-2/AM to the BAY 44-4400 solution or to the PF1022-001, for the remaining 30 min. After the cells had been introduced into the apparatus, 75 nM α-LTX were also added thereto 6 min after starting the experiment. The optimal α-LTX concentration of 75 nM was tested on HEK-293 cells which express the β₂-adrenergic receptor with C-terminal GFP tag (β₂-R-GFP). CdCl₂ (in each case 1 μM and 10 μM) was dissolved in Na⁺ HBS, and nifedipine (15 μM) and EGTA (2 mM) were dissolved in 0.1% DMSO. Addition of CdCl₂ and nifedipine took place immediately at the start of the experiment and before addition of α-LTX, and EGTA was added both at the start of the experiment and 4 min after the addition of α-LTX. Dissolved α-LTX was present in a concentration of 300 nM in 50 mM tris-HCl, pH 8.0; BAY 44-4400 was initially dissolved in pure DMSO (stock solutions between 0.004 and 10 μg/ml). The stock solutions were adjusted to the required concentration in the Na⁺ HBS buffer. The maximum amount of the active substance BAY 44-4400 which could be dissolved at a final concentration of 0.1% DMSO was found by setting up serial dilutions in Na⁺ HBS. Neither 0.1% DMSO nor any other test component interacted with Fura-2/AM at the chosen concentrations.

The data were analyzed using the Excel 98 table calculation programme. The results are derived from at least 2-4 reproduced experiments on in each case 4-8 transfected and un-transfected cells.

The intracellular calcium concentration was calculated by the method of Grynkiewicz et al. (1985) after previous calibration by the method of McCormack et al. (1991).

25. Binding of PF1022A to HC110-R

The binding of PF1022A and the morpholine derivative BAY44-4400 to HC110-R is to be shown by SDS-PAGE, ligand precipitation and analytical flow cytometry below. It is additionally intended to check whether PF1022A and its derivatives lead—like α-LTX—to changes in [Ca²⁺]ᵢ in HC110-R-transfected HEK-293 cells or else affect the HC110-R-mediated α-LTX signalling.

Occasionally, antibodies or proteins which give clear signals in immunofluorescence show no reaction on use of the immunoblot method. Since denaturation of proteins is necessary for carrying out SDS-PAGE, there may be destruction of conformation-dependent epitopes. Antibodies or proteins which specifically recognize such epitopes may therefore no longer bind. Thus, HC110-R-specific binding in a Western blot is not identifiable either with the biotinylated active substance PF1022A or with the morpholine derivative BAY44-4400, which is more soluble than PF1022A. BAY44-4400 differs from PF1022A by 2 morpholine residues which are covalently bonded to the phenyl rings of the two D-phenyllactyl radicals of PF1022A. However, if the proteins are renatured in the SDS separating gel using urea, which brings about partial removal of the SDS through abolishing hydrophobic interactions, it is possible to detect HC110-R-specific binding in the Western blot. For this purpose, total protein from untransfected HEK-293 cells and HEK-293 cells which transiently express HC110-R-Myc/His, and from the 54 kDa N terminus and the 21 kDa C terminus of HC110-R which were expressed in E. coli, were fractionated by SDS-PAGE, renatured and blotted in the usual way. Immunodetection took place after incubation with the ligand BAY444400, with a rabbit anti-PF1022A-KLH immune serum and with a goat anti-rabbit IgG HRP antibody. In addition, a partially renatured SDS polyacrylamide gel with the protein fractions of untransfected HEK-293 cells, HEK-293 cells transiently transfected with HC110-R-Myc/His, and the N terminus of HC110-R expressed in E. coli and total protein from H. contortus was blotted, incubated with PF1022A-biotin and detected using streptavidin HRP. Each of the two blots showed a distinct band 116 kDa in size in the HEK-293 cells which stably expressed HC110-R-Myc/His, whereas the lanes with untransfected cells showed no band at this level. In addition, both the morpholine derivative BAY444400 and the biotinylated PF1022A bound to the 54 kDa N terminus of HC110-R, but the 21 kDa C terminus showed no specific PF1022A binding. It was possible to detect with the biotinylated PF1022A a total of 2 bands in the total protein fraction from adult H. contortus nematodes: a band 110 kDa in size and another band about 88 kDa in size. The latter is very probably a biotinylated protein like that of 83 kDa which has already been described for the nematode Heterakis spumosa, especially since, in contrast to the band 110 kDa in size, it is detected even if detection takes place exclusively with streptavidin HRP. On the other hand, HEK-293 cells stably transfected with HC110-R-Myc/His no longer show a band 116 kDa in size with streptavidin HRP alone. It was furthermore possible to preclude nonspecific signals being caused by the goat anti-rabbit IgG HRP secondary antibody alone.

In order to verify these results further, the following ligand precipitation was carried out. 2 mg and 4 mg portions of magnetic Dynal M-280 streptavidin-coupled beads were mixed with 500 μg of biotinylated PF1022A. In a control mixture with 4 mg of Dynal beads, TST buffer was added in place of PF1022A. Excess PF1022A-biotin was removed by magnetic separation before a mixture of the HC110-R terminus and C terminus expressed in E. coli and previously purified by affinity chromatography was added. After renewed magnetic separation to remove unbound HC110-R fragments, one aliquot derived from 2 mg, and one from 4 mg, of Dynal beads was fractionated by SDS-PAGE and blotted. Detection of the precipitate took place via the N-terminally fused His tag with a mouse anti-His IgG and a rabbit anti-mouse IgG HRP antibody. Only the N-terminal 54 kDa region of HC110-R is precipitated by PF1022A, and accordingly the C terminus of HC110-R, which is not bound to PF1022A, is previously removed by magnetic separation. Nonspecific binding of the N or C terminus of HC110-R to the streptavidin-coupled beads was precluded by adding no biotinylated PF1022A to a reaction mixture.

Binding of the ligand PF1022A in vivo to HC110-R was additionally examined by FACS analysis (FIG. 13). For this purpose, HEK-293 cells were transiently transfected with HC110-R-GFP or GFP-HC110-R or else transiently cotransfected with HC110-R-Myc/His and GFP. Untransfected HEK-293 cells, and HEK-293 transiently transfected with β₂-R-GFP or M1-R-GFP were employed as controls. 24 h after the transfection, the cells were incubated with biotinylated PF1022A, and PF1022A-bound cells were detected using streptavidin-phycoerythrin and were finally fixed. A negative control of cells transfected with HC110-R-GFP was incubated only with streptavidin-phycoerythrin without PF1022A-biotin.

The FACScan was used with an excitation wavelength of 488 nm for the fluorescence analysis of in each case 10000 HEK-293 cells for their cell size, granularity and fluorescence staining. Phycoerythrin has—like TRITC—an absorption spectrum which overlaps with EGFP, while the emission spectra are adequately separated, so that both fluorochromes can be excited at only one wavelength. Cell detritus was excluded from the measurement of the fluorescence intensity by setting a ‘gate’ on the main cell population. For quantitative evaluation, the limits were fixed for negative, unstained and positive, GFP-fluorescent cells on the basis of the untransfected and GFP-transfected cells. The 4.6% of green-fluorescent, untransfected HEK-293 cells (autofluorescence) were subtracted from the other GFP-fluorescent samples; and the value for the nonspecific staining of the cells by the phycoerythrin-coupled streptavidin (5.4%) was subtracted from all red-fluorescent cells (Tab. 1).

TABLE 1
Detection of PF1022A binding to HEK-293 cells transfected with
HC110-R by FACScan

| | | Phycoerythrin |
|---|---|---|
| Plasmid | GFP fluorescence | fluorescence |
| Untransfected | 0 ± 0% | 0 ± 0% |
| GFP | 17.0 ± 0.5% | 1.3 ± 0.4% |
| HC110-R-GFP | 10.9 ± 0.2% | 4.5 ± 0.4% |
| GFP-HC110-R | 9.4 ± 2.5% | 2.5 ± 0.3% |
| β₂-R-GFP | 7.6 ± 0.4% | 0.8 ± 0.1% |
| M1-R-GFP | 8.2 ± 0.7% | 1.6 ± 0.1% |
| HC110-R-Myc/His + GFP | 23.1 ± 2.3% | 10.3 ± 0.7% |

Calculation of the percentage of green-fluorescent cells which were also red-fluorescent after subtraction of the autofluorescence and the nonspecific red staining by streptavidin-phycoerythrin showed that 41.2% of the cells expressing HC110-R-GFP, 26.8% of the cells expressing GFP-HC110-R and 44.5% of the cells cotransfected with HC110-R-Myc/His and GFP bind PF1022A, whereas only 7.5% of the cells expressing GFP, 9.9% of the cells expressing 2-R-GFP and 19.2% of the cells expressing M1-R-GFP bind PF 1022A (FIG. 13).

26. Interactions of PF1022A Derivatives with HC110-R-mediated α-LTX Signal Transduction

PF1022A shows in vitro neuropharmacological activity between 10-9 and 10-3 mg/ml depending on the nematode species. In order to detect any interference of PF1022A and its derivatives with HC1110-R and the α-LTX signalling mediated by HC110-R, the Ca imaging method was used to investigate the effect of BAY44-4400, a more soluble morpholine variant of PF1022A, on HEK-293 cells transiently or stably expressing HC110-R. The optical antipode to PF1022A, PF1022-001, which has an activity which is more than 100 times less in vitro and in vivo was employed as control. BAY44-4400 and PF1022-001 were initially dissolved in pure DMSO because of their hydrophobicity. The stock concentration was chosen so that there was always only 0.1% DMSO present in the experimental mixture. It was possible in control experiments to preclude DMSO concentrations up to and including 0.1% leading to an impairment of the experimental results in untransfected HEK-293 cells and HEK-293 cells transfected with HC110-R-GFP (FIG. 14A). Fura-2-loaded untransfected cells and cells transfected with HC110-R-GFP were stimulated on the one hand with 100, 300 or 400 ng/ml and 1 or 10 μg/ml BAY44-4400 and on the other hand with the same concentrations of PF1022-001. At no concentration was a Ca²⁺-mediated response detectable over the entire period of 50 min. FIGS. 14B and C show by way of example the stimulation of cells transfected with HC110-R-GFP and M1-R-GFP with 400 ng/ml BAY444400 and PF1022-001. Since the PF1022A derivatives are possibly also taken up by the cells and thus may intervene secondarily in signal transduction pathways, and because the PF1022A derivatives remain for a prolonged period in the worm, depending on the parasite, the cells were preincubated with the derivatives for 90 min in some experiments. Cells preincubated with 4 ng/ml or 400 ng/ml BAY44-4400 or PF1022-001 for 90 min showed no change in [Ca²⁺]ᵢ (FIG. 14D). On direct addition of concentrations of 300 ng/ml BAY44-4400 and above, it was possible—in contrast to the optical antipode—to observe on a monitor which represented the cells with 40× magnification a slight swelling of the cells which was, however, reversible within a few minutes, as direct effect of the anthelmintic in HEK-293 cells transfected with HC110-R-GFP.

Very recent experiments have shown that piperazin synergistically enhances the effect of PF1022A/BAY44-4400 in experiments with Heterakis spumosa in vitro and in vivo. When 400 ng/ml BAY44-4400 was given simultaneously with 1 or 10 μM piperazine (FIG. 14F), no change in [Ca²⁺]ᵢ was detectable either in untransfected HEK-293 cells or in HEK-293 cells transiently transfected with HC110-R-GFP. The control mixture with 10 μM piperazine likewise led to no change in the Ca²⁺ balance in untransfected and HC110-R-GFP-transfected cells (FIG. 14E).

In contrast to this, both BAY44-4400 and the optical antipode PF1022-001 influenced, in the presence of 75 nM α-LTX, the α-LTX-induced signal transduction of HC110-R-GFP-expressing HEK-293 cells, although the extent varied (FIGS. 14B-E). In a control experiment in which cells transfected with HC110-R-GFP or M1-R-GFP were treated with 75 nM α-LTX, 0.1% DMSO, 6 min before the stimulation, it was possible to preclude an effect on the α-LTX-induced change in [Ca²⁺]ᵢ exerted by the solvent used for BAY444400 and PF1022-001 at the latter concentration. HC110-R-GFP-expressing cells showed, in contrast to the M1-R-GFP-expressing cells, the previously described biphasic profile with comparable values of [Ca²⁺]ᵢ after addition of α-LTX (FIG. 15A). Incubation of HC110-R-GFP-expressing cells with 4 ng/ml BAY44-4400 6 min before addition of α-LTX diminished the described effect of α-LTX on the Ca²⁺ concentration: the first small increase in [Ca²⁺]ᵢ induced by α-LTX disappeared and the second delayed peak was reduced to 44±6.0 nM Ca²+14 min after the addition of α-LTX (FIG. 15B). In the presence of 4 ng/ml PF1022-001—the addition took place 6 min before stimulation with α-LTX—there was a rapid increase of 103±11.5 nM Ca²⁺, (FIG. 15B). On addition of 400 ng/ml BAY44-4400 to HC110-R-GFP-expressing cells 6 min before the addition of α-LTX there was—like with 4 ng/ml BAY44-4400 (FIG. 15B)—a delayed increase in [Ca²⁺]ᵢ by 49±4.2 nM after 23 min, which fell 8 min later to a plateau which was slightly elevated by 11±1.4 nM compared with the initial values (FIG. 15C). On addition of 400 ng/ml PF1022-001 6 min before incubation with 75 nM α-LTX there was a rapid increase in Ca²⁺, of 112+14.3 nM Ca²⁺ (FIG. 15C), as previously observed on addition of 4 ng/ml PF1022-001 (FIG. 15B). In another design of experiment, HC110-R-GFP-expressing HEK-293 cells were in each case preincubated with 4 ng/ml (FIG. 15D) or 400 ng/ml BAY44-4400 or PF1022-001 (FIG. 15E) for 90 min. Active substance which had not been bound or taken up was carefully washed out after the incubation and loading of the cells with Fura-2/AM before the cells were stimulated with 75 nM α-LTX. HC110-R-transfected cells preincubated with the optical antipode PF1022-001 showed an increase in [Ca²⁺]ᵢ of 102±6.4 nM (FIG. 15D) with 4 ng/ml PF1022-001 and a comparable increase of 109±8.4 nM Ca²⁺ (FIG. 15E) with 400 ng/ml. This increase in Ca²⁺, which was observed only a few minutes after addition of α-LTX, resembled the rapid changes in [Ca²⁺]ᵢ previously observed in FIGS. 15B and C when PF1022-001 was added immediately before stimulation with α-LTX. By contrast, BAY44-4400 showed different responses in relation to the α-LTX-induced Ca²⁺ influx: on preincubation of HC110-R-GFP-expressing cells with 4 ng/ml BAY44-4400 for 90 min, α-LTX induction led to a maximum increase in [Ca²⁺]ᵢ of 95±20.5 nM at 10 min before it fell to an extensive plateau elevated by 23±2.6 nM Ca²⁺ (FIG. 15D). On preincubation with 400 ng/ml BAY44-4400, α-LTX stimulation was followed by an increase in [Ca²⁺]ᵢ by a maximum of 65+7.5 nM (FIG. 15D), which was moreover delayed by 12 min compared with preincubation with 4 ng/ml BAY44-4400 (FIG. 15C).

On addition of 400 ng/ml BAY44-4400 to HEK-293 cells stably expressing HC110-R-Myc/His 6 min before addition of α-LTX there is complete inhibition of the observed immediate increase in [Ca²⁺]ᵢ by 267+45.8 nM (FIG. 15F). In contrast to this, addition of 400 ng/ml PF1022-001 leads to no significant change in [Ca²⁺]ᵢ, the increase in Ca⁺ being 209±33.4 nM Ca (FIG. 15F).

In order to show the specific effect of BAY44-4400 on the α-LTX-mediated Ca²⁺ signalling in HC110-R-expressing cells, the following control experiments were carried out: initially untransfected HEK-293 cells were stimulated with 1 mM carbamylcholine chloride (carbachol), a ligand for muscarinic acetylcholine receptors, in the presence and absence of 400 ng/ml BAY44-4400 (FIG. 15G). In both cases there was a comparable immediate increase in [Ca²⁺]ᵢ by endogenous, natural muscarinic acetylcholine receptors by 58±8.1 nM Ca²⁺ for HEK-293 cells in the presence of BAY44-4400 and by 53+6.8 nM Ca²⁺ in the absence of the anthelmintic. Additional transient transfection of HEK-293 cells with the human muscarinic M1 acetylcholine receptor C-terminally fused to GFP (M1-R-GFP) led to a slight increase in [Ca 2+], compared with untransfected cells (FIG. 15G), by 89±5.5 nM in the presence and by 85±6.1 nM Ca²⁺ in the absence of 400 ng/ml BAY44-4400 (FIG. 15H). Nor was any significant effect of BAY44-4400 on M1-R-GPCR observable.

Stimulation of the endogenously present natural β₂-adrenergic receptors in untransfected HEK-293 cells with 1 mM isoproterenol, or the nicotinic receptors with 1 mM arecoline—as previously described for carbachol—in the presence and absence of 400 ng/ml BAY44-4400 once again led to no signficant difference in [Ca²⁺]ᵢ (FIG. 15I). Isoproterenol induced an increase of 45±4.7 nM in [Ca²⁺]ᵢ in the presence and an increase of 48±5.7 nM Ca²⁺ in the absence of BAY44-4400. Following arecoline stimulation there was an increase of 28±4.1 nM in Ca²⁺ in the presence of BAY44-4400 and of 27±3.5 nM in Ca²⁺ in the absence of the active substance (FIG. 151).

27. Obtaining Antibodies

Antibodies were obtained by employing female NMRI mice and rabbits (chinchilla crosses). For immunization of the NMRI mice, 3×15 μg of the purified 21 kD C-terminal HC110-R protein fragment were each dissolved in 100 μl of PBS⁻ together with 100 μl of FCA and injected subcutaneously into two naïve NMRI mice on days 1, 8 and 15. Blood sampling and obtaining of serum took place on day 23.

To obtain serum, the blood obtained by cardiac puncture was incubated initially at 37° C. for 1 h and then at 4° C. for at least 2 h. The cellular constituents were subsequently pelleted twice at 3 000 rpm and 4° C. in the Beckman GPKR centrifuge for 10 min, and the supernatant was inactivated at 56° C. for 45 min. After a further centrifugation at 13 000 rpm and 4° C. in the Heraeus biofuge 15R for 10 minutes it was possible to take off the serum and store it at −20° C. until used further.

After the first blood sampling and obtaining of preimmune serum, two rabbits initially received subcutaneous injections of 50 μg of the C-terminal 21 kD HC110-R protein and of the 54 kD N-terminal HC110-R protein in a suspension consisting of equal parts of PBS⁻ and FCA. Two further immunizations each with 100 μg of antigen took place on day 31 and day 79 after the first immunization. A first blood sampling was carried out on day 43. A second blood sampling and obtaining of serum took place on day 98 after the first immunization.

### DESCRIPTION OF THE FIGURES

FIG. 1: HC110-R mRNA and protein
(A) Northern blot analysis using total RNA from Haemonchus contortus and the 3.6 kbp cDNA of HC110-R.
(B) 10% SDS-PAGE fluorogram of in vitro translated ³⁵S-labelled HC110-R protein. In vitro translation of 1 μg of in vitro transcribed HC110-R mRNA (HC110-R), negative control without HC110-R mRNA (control), 1 μg of luciferase mRNA as positive control (luciferase).

FIG. 2: Full-length cDNA sequence of HC110-R and derived amino acid sequence

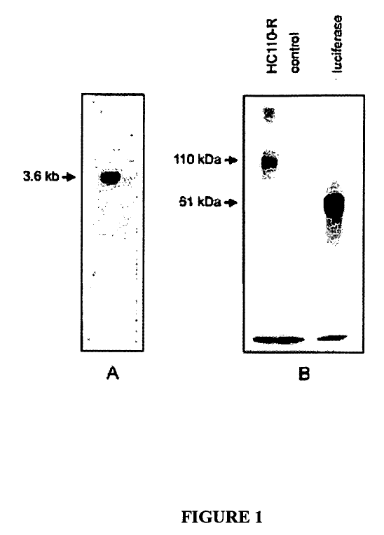

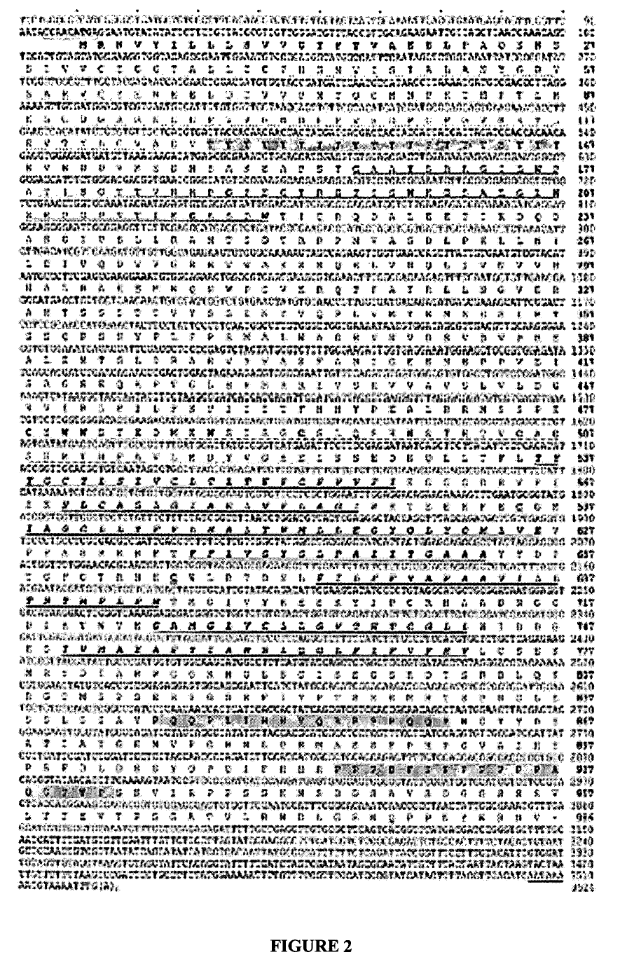

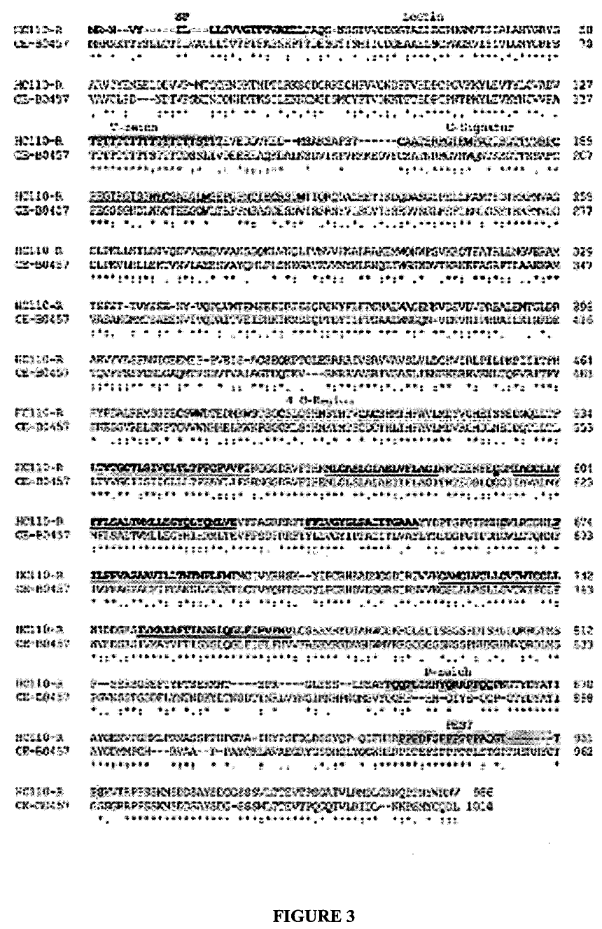

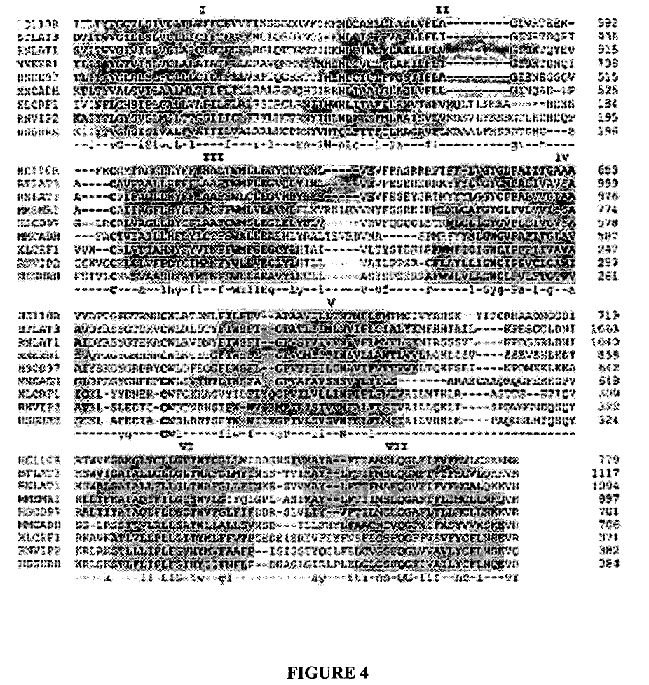

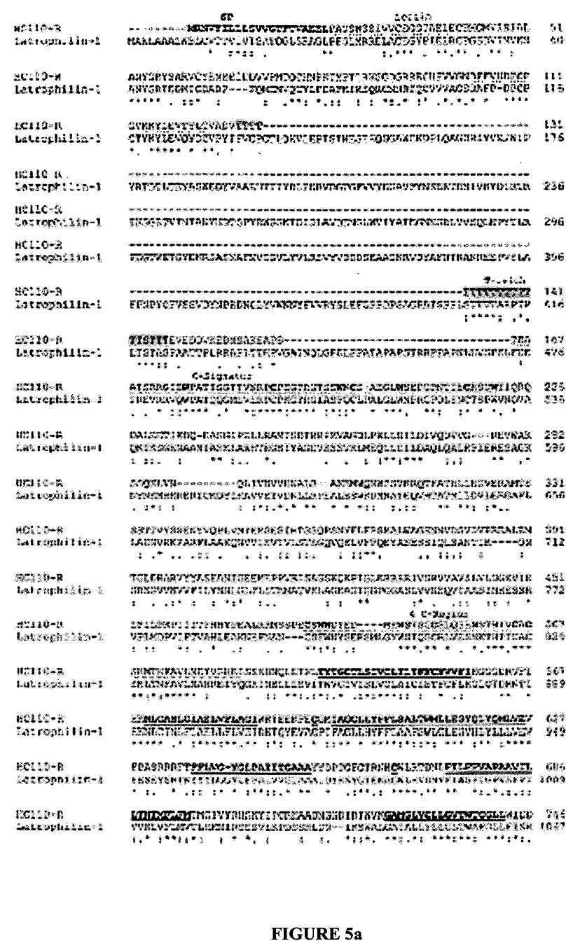

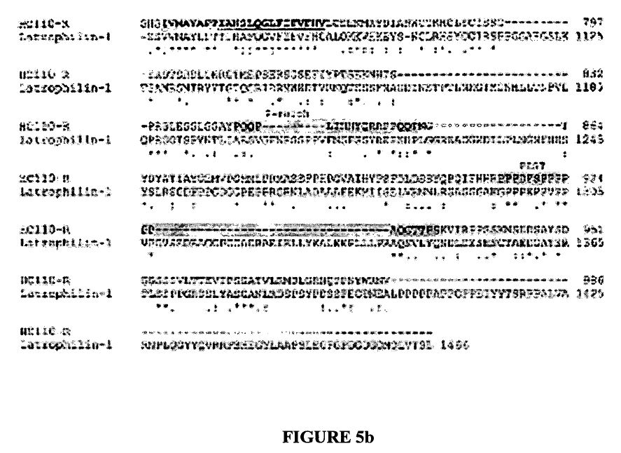

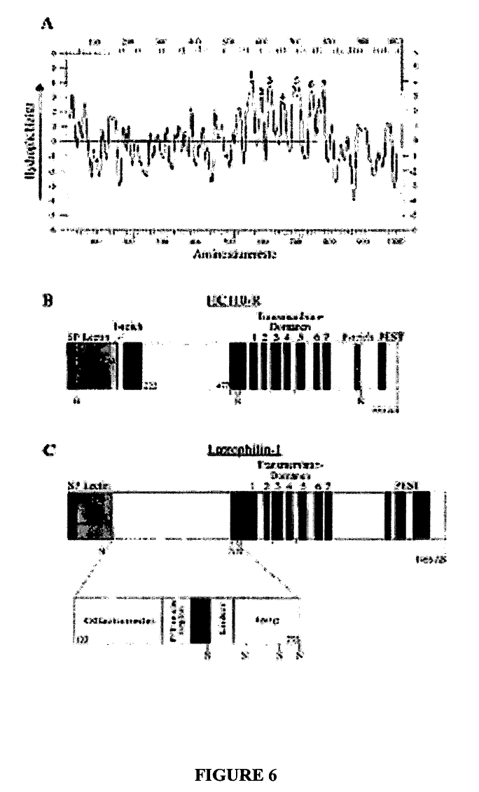

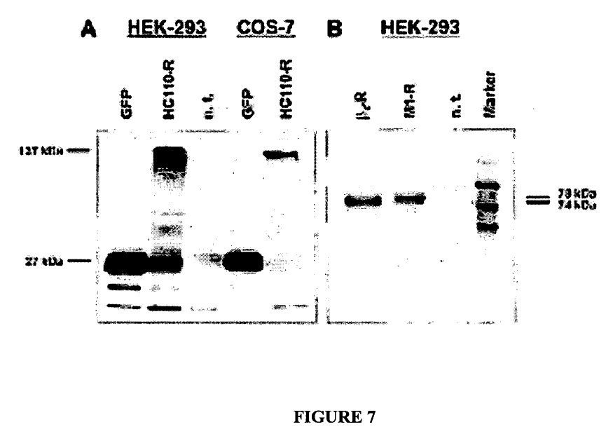

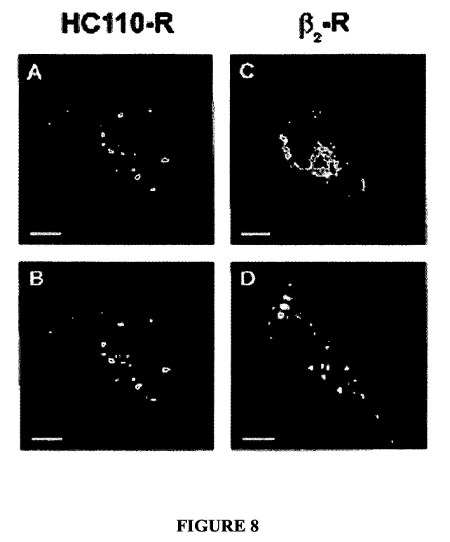

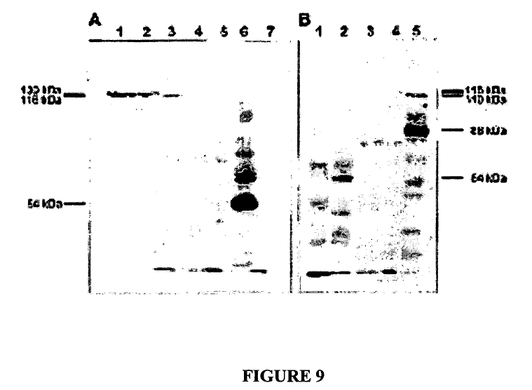

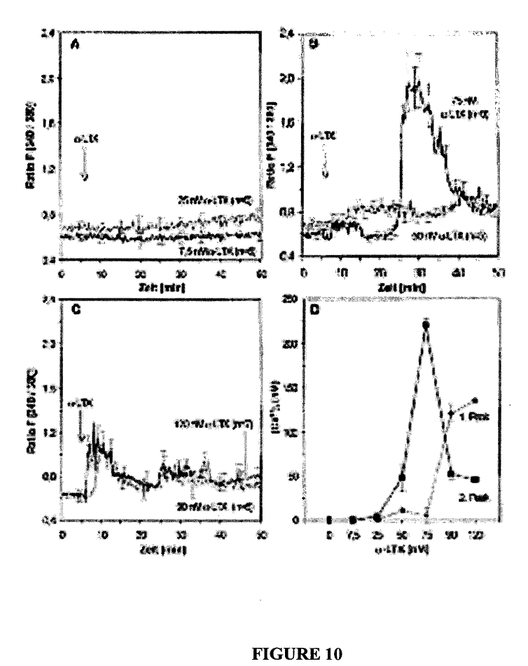

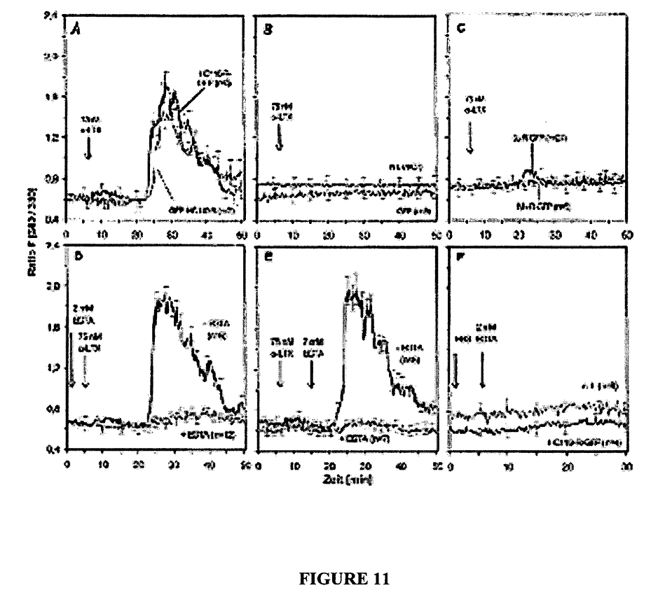

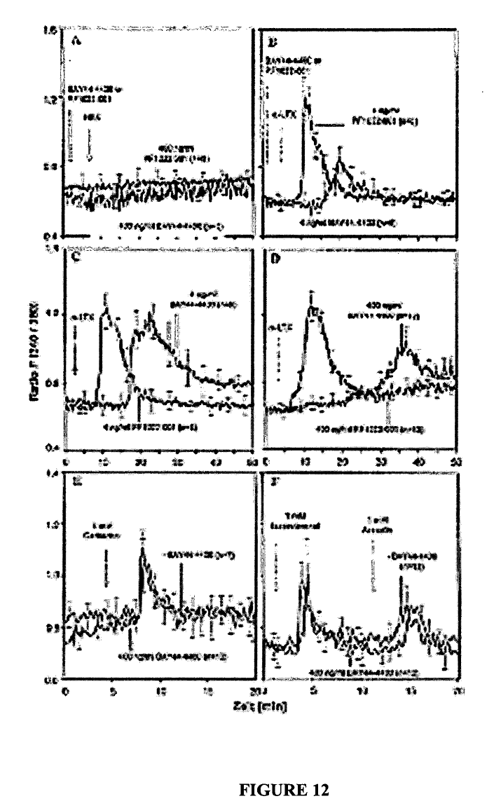

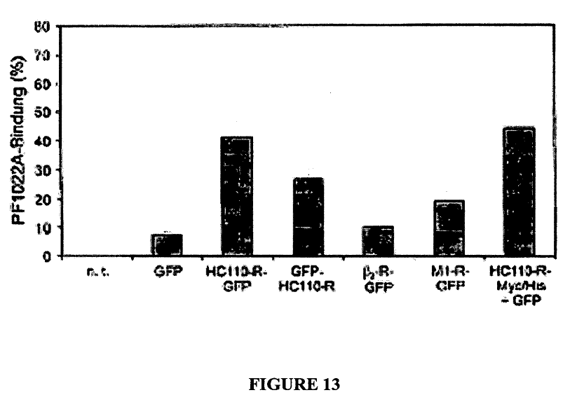

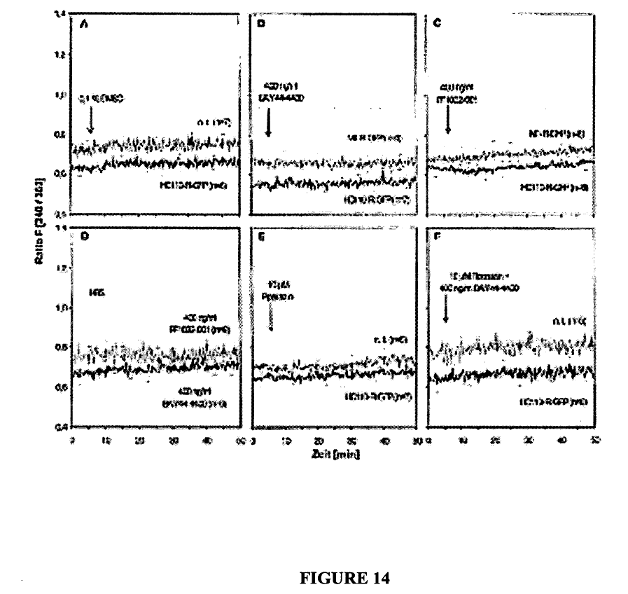

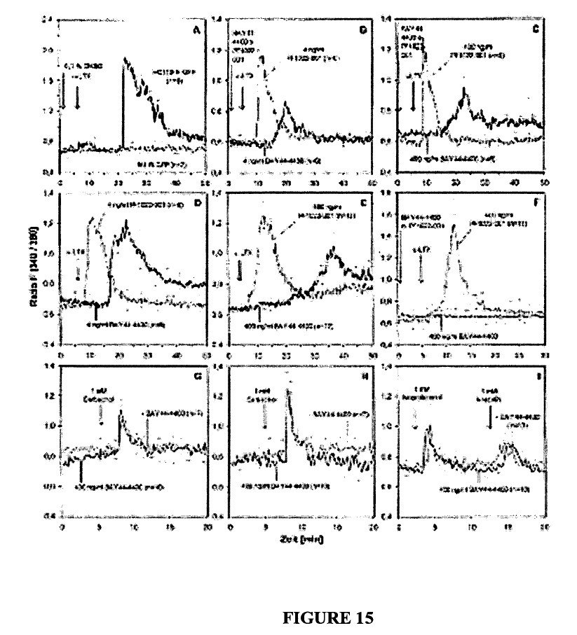

The Kozak motif (Kozak, 1989) and the polyadenylation signal of HC110-R (GenBank Accession No.: AJ272270) are underlined; the start codon in position 100 is emboldened. Signal peptide (residues 1-21, bold), lectin-like sequence (residues 22-125, dotted), Thr stretch (residues 128-147, grey background); cysteine-rich region of structure CX₉WX₁₂CX₉WXCX₅WX₉CX₃W (residues 166-221, wavy line, Cys and Trp residues additionally bold); 4-Cys region of structure CXWWX₆WX₄CX₁₁CXC (residues 478-524, broken line, Cys and Trp residues additionally bold); the 7 transmembrane regions (between residues 563-772, bold and underlined); the proline-rich stretch (residues 845-861, grey background), the PEST region (residues 915-933, grey background); the N-glycosylation sites (residues 26, 499 and 862, bold); the highly conserved putative disulphide linkage of the Cys-Cys pair between secretin GPCRs (position 595 and 666, bold and double underlining).

FIG. 3: Alignment of the derived amino acid sequences of the H. contortus HC110-R and Caenorhabditis elegans cosmid clone B0457 (CE-B0457; GenBank Accession No.: Z54306)

Signal peptide (SP, residues 1-21, bold), lectin-like sequence (lectin, residues 22-125, dotted); Thr stretch (T-rich, residues 128-147, grey background); Cys-rich region of structure CX₉WX₁₂CX₉WXCX₅WX₉CX₃W (C signature, residues 166-221, wavy line, Cys and Trp residues additionally bold); 4-Cys motif of structure CXWWX₆WX₄CX₁₁CXC (4 C region, residues 478-524, broken line, Cys and Trp residues additionally bold); the 7 transmembrane regions (between residues 536-772, bold and underlined); the Pro-rich stretch (P-rich, residues 845-861, grey background); the PEST region (PEST, residues 915-933, grey background); the N-glycosylation sites (residues 26, 499 and 862, bold); the highly conserved putative disulphide linkage of the Cys-Cys pair between secretin GPCRs (position 595 and 666, bold and double underlining). Identical amino acids are marked with asterisks, very closely related amino acids with a colon, related amino acids with a single dot.

FIG. 4: Alignment of the 7 transmembrane receptor HC110-R and several other receptors of the secretin subfamily

HC110-R: Haemonchus contortus heptahelical orphan transmembrane receptor (Accession no.: AJ272270); BTLAT3: Bos taurus latrophilin-3 (several splicing variants, Accession no.: G4164053-G4164075); RNLAT1: Rattus norvegicus latrophilin-1 (Accession no.: U78105, U72487); MMEMR1: Mus musculus EMR1 hormone receptor precursor (Accession no.: Q61549); HSCD97: Homo sapiens leukocyte activation antigen CD97 (Accession no.: P48960); MMCADH: M. musculus cadherin 7 transmembrane receptor precursor (Accession no.: G3800738); XLXRF1: Xenopus laevis corticotropin releasing receptor precursor (Accession no.: 042602); RNVIP1: R. norvegicus vasoactive intestinal polypeptide receptor precursor 2 (Accession no.: Q02643). The seven transmembrane domains have a grey background (I-VII) and the highly conserved putative disulphide linkage is emboldened.

FIG. 5: Alignment of the derived amino acid sequences of the H. contortus HC 110-R and R. norvegicus latrophilin-1 (GenBank Acc. No. U78105, U72487)

Signal peptide (SP, residues 1-21, bold), lectin-like sequence (lectin, residues 22-125, dotted); Thr stretch (T-rich, residues 128-147, grey background); Cys-rich region of structure CX₉WX₁₂CX₉WXCX₅WX₉CX₃W (C signature, residues 166-221, wavy line, Cys and Trp residues additionally bold); 4-Cys motif of structure CXWWX₆WX₄CX₁₁CXC (4 C region, residues 478-524, broken line, Cys and Trp residues additionally bold); the 7 transmembrane regions (between residues 536-772, bold and underlined); the Pro-rich stretch (P-rich, residues 845-861, grey background); the PEST region (PEST, residues 915-933, grey background); the N-glycosylation sites (residues 26, 499 and 862, bold); the highly conserved putative disulphide linkage of the Cys-Cys pair between secretin GPCRs (position 595 and 666, bold and double underlining). Identical amino acids are indicated by asterisks, very closely related amino acids by a colon related amino acids by a single dot.

FIG. 6: Structure of the HC110-R proteins and of the rat latrophilin-1
(A) Hydrophobicity blot of HC110-R with the 7 transmembrane domains.
(B) Protein structure of HC110-R with the following characteristic motifs: signal sequence (SP), lectin domain (lectin), Thr stretch (T-stretch), Cys motif (signature), 4-Cys region (4C region), 7 transmembrane domains (1-7, black), the Pro-rich motif (P-rich) and the PEST sequence (PEST). The putative glycosylation sites are depicted underneath the diagram (N), as are the Cys residues of the 4C region and the two conserved Cys residues of the putative disulphide linkage (thin line without capital letters).
(C) Protein structure of latrophilin-1. Latrophilin-1 has no Thr stretch but contains an additional olfactomedin binding motif (olfactomedin), a Pro-Thr region (P-T region), a linker domain (linker) and a long region containing several repeats (long).

FIG. 7: Expression of HC110-R-GFP in HEK-293 and COS-7 cells
(A) Total protein from HEK-293 and COS-7 cells transiently transfected with GFP (GFP) or HC110-R-GFP (HC110-R) and untransfected (n. t.) HEK-293 and COS-7 cells was isolated by the Trizol method 24 h after transfection.
(B) Total protein from HEK-293 cells transiently transfected with β₂-R-GFP (β2-R) or M1-R-GFP (M1-R), and untransfected (n. t.) HEK-293 cells was isolated by the Trizol method 24 h after the transfection. SDS marker bands at 116, 90, 70 and 55 kDa (marker).

20 μg of total protein per lane were fractionated on a 10% SDS polyacrylamide gel and blotted onto a nitrocellulose membrane. Blocking in RotiBlock solution overnight was followed by immunodetection using the monoclonal mouse anti-GFP IgG primary antibody (0.4 μg/ml) and the monoclonal rabbit anti-mouse IgG-HRP secondary antibody (1:25000) by the ECL system.

FIG. 8: Cellular localization of HC110-R and β₂-R in transfected COS-7 cells COS-7 cells were transfected with HC110-R-GFP and β₂-R-GFP both of which carry a C-terminal GFP tag. The CLSM was carried out 24 hours after the transfection. The bars correspond to 10 μm.
(A) HC110-R-GFP is expressed on the plasma membrane and in the cytoplasmic compartments. A compaction of cytoplasmic vesicles can be seen in the vicinity of the nucleus.
(B) CLSM colocalization of the green HC110-R-GFP protein with acidic lysosomes, stained with LysoTracker Red DND-99 at 37° C. for 1 h.
(C) β₂-R-GFP-transfected cells show a similar GFP fluorescence pattern to HC110-R-GFP, or as described under (A). The receptor can be localized to the plasma membrane and in vesicles and can in some cases be colocalized with acidic lysosomes, stained with LysoTracker Red DND-99 at 37° C. for 1 h.
(D) Expressed β₂-R-GFP colocalizes in some cases with the endoplasmic reticulum, stained with monoclonal anti-KDEL antibodies.

FIG. 9: Binding of α-latrotoxin to HC110-R

(A) 5 μg of total protein from isolated Latrodectus revivensis venom glands (1), and pure 130 kDa (X-LTX (2), also in each case 40 μg of total protein from HEK-293 cells stably transfected with the HC110-R-Myc/His construct in the pIRES1neo vector (3), untransfected cells (4) and the 54 kDa N terminus (6) and 21 kDa C terminus (7) of HC110-R induced in E. coli were fractionated in a 10% SDS polyacrylamide gel, blotted and blocked overnight. The denatured proteins were incubated with 20 nM α-LTX for 2 h and possible bindings of α-LTX detected with a rabbit anti-α-LTX IgG-HRP (1:5000), a goat anti-rabbit IgG-HRP antibody (1:25000) and the ECL system.
(B) In each case 40 μg of total protein from the C terminus (1) and N terminus (2) of HC110-R induced in E. coli, and untransfected HEK-293 cells (3) and HEK-293 cells stably transfected with HC110-R-Myc/His (4) and adult H. contortus nematodes (5) were fractionated in a 10% SDS polyacrylamide gel and partially renatured with a 4 M urea-containing renaturation buffer. The blot was blocked overnight, incubated with 20 nM pure α-LTX, and α-LTX-binding proteins were detected with a rabbit anti-α-LTX IgG antibody (1:5000), biotin-protein A (1:100), streptavidin-peroxidase (1:3000) and the ECL system.

FIG. 10: Dose-dependence of α-LTX on the HC110-R-mediated Ca²⁺ signalling

48 h after transient transfection with HC110-R-GFP, HEK-293 cells were loaded with 1 μM Fura-2/AM for 30 min. The cells were stimulated 6 min after the start of the measurement with various α-LTX concentrations for a further 44 min. The 340/380 nm quotient (ratio) was measured as a function of the time. The number of selected cells is indicated by n.
(A) HC110-R-GFP-expressing cells were stimulated with 7.5 nM (black line) or 25 nM α-LTX (grey line).
(B) 50 nM (grey line) or 75 nM α-LTX (black line) was added to HC110-R-GFP-expressing cells.
(C)HC110-R-GFP-expressing cells were stimulated with 90 nM (grey line) or 120 nM α-LTX (black line).
(D) [Ca²⁺]ᵢ is plotted in nM on the abscissa, and the α-LTX concentrations (0, 7.5, 25, 50, 75, 90 and 120 nM) for the first rapid peak (1st peak, grey line) and the second delayed peak (2nd peak, black line) employed to stimulate HC110-R-GFP-expressing HEK-293 cells are plotted on the ordinate.

FIG. 11: Ca²⁺ Imaging of HC110-R-transfected HEK 293 cells
(A) HC110-R with C-terminal GFP tag (HC110-R-GFP): black line.
   HC110-R With N-terminal GFP tag (GFP-HC110-R): grey line.
(B) 75 nM α-LTX with untransfected HEK-293 cells (n. t., black line) and GFP-transfected cells (GFP, grey line).
(C) 75 nM α-LTX with cells transfected with C-terminally tagged human M1 muscarinic acetylcholine receptor (M1-R-GFP, grey line) and with C-terminally tagged mouse β₂-adrenergic acetylcholine receptor (β₂-R-GFP, black line).
(D) HC110-R-GFP-transfected cells were treated with 2 mM EGTA 6 minutes before addition of α-LTX (75 nM) (+EGTA, grey line). EGTA was omitted from the control (−EGTA, black line).
(E) 2 mM EGTA were added 10 min after addition of α-LTX (75 nM) (+EGTA, grey line). No EGTA was added to the control mixture (−EGTA, black line).
(F) As controls, untransfected (n. t., grey line) and HC110-R-GFP-expressing cells (black line) were mixed with 2 nM EGTA for 30 minutes.

FIG. 12: Interference by BAY44-4400 with signal transmission caused by α-LTX

HEK-293 cells were transiently transfected with GFP-tagged HC110-R protein and stimulated (arrows) 48 h after the transfection. Ca²⁺ imaging was carried out for 50 minutes (Figs. A-D), control experiments with various endogenous natural receptors of untransfected HEK-293 cells for 20 minutes. n=number of cells.
(A) Addition of 400 ng/ml BAY44-4400 (black line) or of PF1022-001 (grey line) to the cells. As controls, the cells were treated with Na⁺ HBS/HEPES buffer (HBS), which also served as solvent for the respective substances, after the adddition of BAY44-4400 or PF1022-001.
(B) Signal transmission caused by α-LTX (75 nM) in the presence of 4 ng/ml BAY44-4400 (black line) and PF1022-001 (grey line).
(C) Signal transmission caused by α-LTX in cells preincubated with 4 ng/ml BAY44-4400 (black line) or PF1022-001 (grey line) for 90 minutes. The substances were removed before addition of α-LTX.
(D) Signal transmission caused by α-LTX in HC110-R-GFP-transfected cells preincubated with 400 ng/ml BAY44-4400 (black line) or PF1022-001 (grey line) for 90 minutes. The substances were removed before addition of α-LTX.
(E) Untransfected HEK-293 cells were stimulated with 1 mM carbamylcholine chloride (carbachol) for 100 s in the presence (400 ng/ml BAY44-4400, black line) or absence of 400 ng/ml BAY44-4400 (grey line, -BAY44-4400).
(F) Untransfected HEK-293 cells were stimulated with 1 mM isoproterenol and with 1 mM arecoline in each case for 100 s in the presence (400 ng/ml BAY 44-4400, black line) or absence of 400 ng/ml BAY44-4400 (-BAY44-4400, grey line).

FIG. 13: Detection of PF1022A binding to HC110-R-transfected HEK-293 cells by FACScan

In each case 5×10⁵ HEK-293 cells which were not transfected, were transiently transfected with GFP, HC110-R-GFP, GFP-HC110-R, β₂-R-GFP and M1-R-GFP, and transiently cotransfected with HC110-R-Myc/His and GFP were incubated, 24 h after the transfection, with 0.5 μg/ml PF1022A-biotin and streptavidin-phycoerythrin (1:300) and then fixed. As negative controls, HC110-R-GFP-transfected cells were incubated only with streptavidin-phycoerythrin, without PF1022A-biotin. After subtraction of the autofluorescence, i.e. of the 4.6% untransfected cells (n. t.) in the green channel from all green-fluorescent cells and of the 4.4% nonspecific staining caused by streptavidin-phycoerythrin in the red channel, the percentage of GFP-fluorescent cells (GFP, HC110-R-GFP, GFP-HC110-R, β₂-R-GFP, M1-R-GFP and HC110-R-Myc/His+GFP) which have bound PF1022A was found (PF1022A binding in %). The standard deviation is derived by determining the means of triplicate mixtures.

FIG. 14: Effect of BAY44-4400 and the optical antipode PF1022-001 on the HC110-R-mediated Ca²⁺ signalling

Loading of the cells with 1 μM Fura-2/AM took place in untransfected HEK-293 cells and 48 h after transient transfection with HC110-R-GFP or M1-R-GFP. The quotient of 340/380 nm (ratio) was measured as a function of the time. The number of selected cells is indicated by n.
(A) Untransfected (n. t., grey line) and HC110-R-GFP-transfected (HC110-R-GFP, black line) cells were mixed with 0.1% of the solvent DMSO in Na⁺ HBS buffer after 6 min as control.
(B) HC110-R-GFP- (black line) and M1-R-GFP- (grey line) transfected cells were stimulated with 400 ng/ml BAY44-4400 after 6 min.
(C) As (B), HC110-R-GFP- (black line) and M1-R-GFP- (grey line) transfected cells were stimulated with 400 ng/ml PF1022-001 after 6 min.
(D) HC110-R-GFP-expressing cells were preincubated with 400 ng/ml BAY44-4400 (black line) or PF1022-001 (grey line) for 90 min, and unbound substances were washed out before the measurement.
(E) 10 μM piperazine were added to untransfected (n. t., grey line) and HC110-R-GFP-expressing cells (HC110-R-GFP, black line) after 6 min.
(F) As (E), but a mixture of 10 μM piperazine and 400 ng/ml BAY44-4400 was administered to untransfected (n. t., grey line) and HC110-R-GFP-expressing cells (HC110-R-GFP, black line) after 6 min.

FIG. 15: Interactions of the HC110-R-mediated α-LTX signalling through PF1022A derivatives

Loading of the cells with 1 μM Fura-2/AM took place in untransfected HEK-293 cells and 48 h after transient transfection with HC110-R-GFP or M1-R-GFP. The amount of α-LTX employed was always 75 nM. For the experiments with the control substances, the cells were flushed at a flow rate of 1.6 ml Na⁺ HBS/min. The quotient of 340/380 nm (ratio) was measured as a function of the time. The number of selected cells is indicated by n.
(A) HC110-R-GFP- (black line) and M1-R-GFP- (grey line) expressing cells were mixed with 0.1% DMSO 6 min before α-LTX stimulation in order to preclude DMSO having an effect on α-LTX-induced changes in [Ca²⁺]ᵢ.
(B) HC110-R-GFP-transfected cells were mixed with 4 ng/ml BAY44-4400 (black line) or PF1022-001 (grey line) 6 min before addition of α-LTX.
(C) As (B), but 400 ng/ml BAY44-4400 (black line) or PF1022-001 (grey line) were added to HC110-R-GFP-expressing cells 6 min before stimulation with α-LTX.
(D) HC110-R-GFP-transfected cells were preincubated with 4 ng/ml BAY44-4400 (black line) or PF1022-001 (grey line) for 90 min, and unbound substances were removed before the measurement. Stimulation of the cells took place after 6 min with α-LTX.
(E) As (D), but HC110-R-GFP-transfected cells were preincubated with 400 ng/ml BAY44-4400 (black line) or PF1022-001 (grey line) for 90 min, unbound substances were removed before the measurement, and the cells were stimulated with α-LTX after 6 min.
(F) Cells stably expressing HC110-R-Myc/His were mixed with 400 ng/ml BAY44-4400 (black line) or PF1022-001 (grey line) 6 min before addition of α-LTX.
(G) As controls, 1 mM carbamylcholine chloride (carbachol) was added after 6 min for 100 s, and washed out again with Na⁺ HBS, to untransfected cells in the presence (400 ng/ml BAY44-4400, black line) or absence (-BAY44-4400, grey line) of 400 ng/ml BAY44-4400.
(H) As (G), but M1-R-GFP-transfected cells were stimulated in the presence (400 ng/ml BAY44-4400, black line) or absence (-BAY44-4400, grey line) of 400 ng/ml BAY44-4400 after 6 min with 0.1 mM carbachol for 100 s and washed out again with Na⁺ HBS.
(I) As controls, untransfected cells were stimulated in the presence (400 ng/ml BAY44-4400, black line) or absence (-BAY44-4400, grey line) of 400 ng/ml BAY44-4400 after 3 min for 100 s with 1 mM isoproterenol and after 12 min for 100 s with 1 mM arecoline. The substances were washed out again with Na⁺ HBS after the 100 s.

### REFERENCES

1. Allard W. J., Sigal I. S. and Dixon R. A. F. (1987) Sequence of the gene encoding the human M1 muscarinic acetylcholine receptor. Nucleic Acids Res. 24, 10604.
2. Altschul S. F. and Lipman D. J. (1990) Protein database searches for multiple alignments. Proc. Natl. Acad. Sci. U.S.A. 87, 5509-5513.
3. Appel R. D., Bairoch A. and Hochstrasser D. F. (1994) A new generation of information retrieval tools for biologists: the example of the ExPASy WWW server. Trends Biochem. Sci. 19, 258-260.
4. Aviv H. and Lederer P. (1972) Purification of biologically active globin messenger RNA by chromatography on oligothymidylic acid-cellulose. Proc. Natl. Acad. Sci. U.S.A. 69, 1408-1412.
5. Bacon K. B., Westwick J. and Camp R. D. (1989) Potent and specific inhibition of IL-8-, IL-1 alpha- and IL-1 beta-induced in vitro human lymphocyte migration by calcium channel antagonists. Biochem. Biophys. Res. Comm. 165, 349-354.
6. Boyd V. L., Bozzini M., Zon G., Noble R. L. and Mattaliano R. J. (1992) Sequencing of peptides and proteins from the carboxy terminus. Anal. Biochem. 206, 344-352.
7. Bradford M. (1976) A rapid and sensitive method for the quantification of microgram quantities of proteins utilizing the principle of protein-dye binding. Anal. Biochem. 72, 248-252.
8. Brendel V., Bucher P., Nourbakhsh I. R., Blaisdell B. E. and Karlin S. (1992) Methods and algorithms for statistical analysis of protein sequences. Proc. Natl. Acad. Sci. U.S.A. 89, 2002-2006.
9. Chalfie M., Tu Y., Euskirchen G., Ward W. W. and Prasher D. C. (1994) Green fluorescent protein as a marker for gene expression. Science 263, 725-888.
10. Chan M-S. (1997) The global burden of intestinal nematode infections—fifty years on. Parasitol. Today 13, 438-443.
11. Chen W., Terada M. and Cheng J. T. (1996) Characterization of subtypes of gamma-aminobutyric acid receptors in an Ascaris muscle preparation by binding assay and binding of PF1022A, a new anthelmintic, on the receptors. Parasitol. Res. 82, 97-101.
12. Chomczynski P. (1992) One-hour downward alkaline capillary transfer for blotting of DNA and RNA. Anal. Biochem. 201, 134-139.
13. Chomczynski P. (1993) A reagent for the single-step simultaneous isolation of RNA, DNA and proteins from cell and tissue samples. BioTechniques 15, 532-534; 536-537.
14. Chomczynski P. and Sacchi N. (1987) Single-step method of RNA-isolation by acid guanidinium thiocyanate-phenol-chloroform extraction. Anal. Biochem. 162, 156-159.
15. Coombs L. M., Pigott D., Proctor A., Eydmann M., Denner J. and Knowles M. A. (1990) Simultaneous isolation of DNA, RNA, and antigenic protein exhibiting kinase activity from small tumor samples using guanidine isothiocyanate. Anal. Biochem. 188, 338-343.
16. Conder G. A., Johnson S. S., Nowakowski D. S., Blake T. E., Dutton F. E., Nelson S. J., Thomas E. M., Davis J. P. and Thompson D. P. (1995) Anthelmintic profile of the cyclodepsipeptide PF1022A in in vitro and in vivo models. J. Antibiotics 48, 820-823.
17. Davletov B. A., Meunier F. A., Ashton A. C., Matsushita H., Hirst W. D., Lelianova V. G., Wilkin G. P., Dolly J. O. and Ushkaryov Y. A. (1998) Vesicle exocytosis stimulated by α-latrotoxin is mediated by latrophilin and requires both external and stored Ca²⁺. EMBO J. 17, 3909-3920.
18. Davletov B. A., Shamotienko O. G., Lelianova V. G., Grishin E. V. and Ushkaryov Y. A. Isolation and biochemical characterization of a Ca²⁺-independent α-latrotoxin-binding protein. J. Biol. Chem. 271, 23239-23245.
19. Drexler H. G., Dirks W., MacLeod R. A. F., Quentmeier H. and Steube K. (eds.) (1995) DSM Catalogue of Human and Animal Cell Lines; Fifth Edition, Braunschweig
20. Jackson R. J., Howell M. T. and Kaminski A. (1990) The novel mechanism of initiation of picornavirus RNA translation. Trends Biochem. Sci. 15, 477-483.
21. Devereux J., Haeberli P. and Smithies O. (1984) A comprehensive set of sequence analysis programs of VAX. Nucleic Acids Res. 12, 387-395.
22. Fivash M., Towler E. M., Fisher R. J. (1998) Biacore for macromolecular interaction. Curr. Opin. Biotechnol. 9, 97-101.
23. Fukashe T., Koike T., Chinone S., Akihama S., Iagake H., Takagi M., Shimizu T., Yaguchi T., Sasaki T. and Okada T. (1990) Anthelmintic effects of PF1022A, a new cyclic depsipeptide, on the intestinal parasitic nematodes in dogs and cats. Proc 110^{th} Meet Jap Soc vet Sci, 122 (Abstr.).
24. Giese G., Wiegers W., Kubbies M., Scherbarth A. and Traub P. (1995) Okadaic acid co-induces vimentin expression and cell cycle arrest in MPC-11 mouse plasmacytoma cells. J. Cell. Physiol. 163, 145-154.
25. Geβner G., Methe S., Rink T. and Boheim G. (1996) Ionophore and anthelmintic activity of PF1022A, a cyclooctadepsipeptide, are not related. Pestic. Sci. 48, 399-407.
26. Grynkiewicz G., Poenie M. M. and Tsien R. Y. (1985) A new generation of Ca²⁺ indicators with greatly improved fluorescence properties. J. Biol. Chem. 260, 3440-3450.
27. Gyuris J., Golemis E., Chertkov H. and Brent R. (1993) Cdi 1, a human G1 and S phase protein phosphatase that associates with Cdk 2. Cell 75, 791-803.
28. Harder A., Londershausen M. and Mehlhorn H. (1997) The four larval stages and the adults of Heterakis spumosa are impaired by the anthelmintic cyclodepsipeptide PF 1022A. Zbl. Bakteriol. 286, 212.
29. Henkel A. W. and Sankaranarayanan S. (1999) Mechanisms of α-latrotoxin action. Cell Tissue Res. 296, 229-233.
30. Holz G. G. and Habener J. F. (1998) Black widow spider alpha-latrotoxin: a presynaptic neurotoxin that shares structural homology with the glucagon-like peptide-1 family of insulin secretagogic hormones, Comp. Biochem. Physiol. B. Biochem. Mol. Biol. 121, 177-184.
31. Hunkapiller M. W., Hewick R. M., Dreyer W. J. and Hood L. E. (1983) High sensitivity sequencing with a gas phase sequenator. Meth. Enzymol. 91, 399-413.
32. Ichtchenko, K., Bittner, M. A., Krasnoperov, V., Little, A. R., Chepurny, O., Holz, R. W. and Petrenko, A. G. (1999) A novel ubiquitously expressed α-latrotoxin receptor is a member of the CIRL family of G-protein-coupled receptors. J. Biological Chem. 274, 5491-5498.
33. Jackson R. J., Howell M. T. and Kaminski A. (1990) The novel mechanism of initiation of picornavirus RNA translation. Trends Biochem. Sci. 15, 477-483.
34. Jang S. K., Krausslich H. G., Nicklin M. J., Duke G. M., Palmenberg A. C. and Wimmer E. (1988) A segment of the 5′ nontranslated region of encephalomyocarditis virus RNA directs internal entry of ribosomes during in vitro translation. J. Virol. 62, 2636-2643.
35. Kachi S., Ishih A. and Terada M. (1994) Effects of PF1022A on Angiostrongylus cantonensis staying in the central nervous system of rats and mice. Jpn. J. Parasitol. 43, 483-488.
36. Kachi S., Ishih A. and Terada M. (1995) Effects of PF1022A on adult Angiostrongylus cantonensis in the pulmonary arteries and larvae migrating into the central nervous system of rats. Parasitol. Res. 81, 631-637.
37. Kachi S., Terada M. and Hashimoto H. (1997) Influence of PF1022A on the motility of Angiostrongylus cantonensis in vitro. Parasitol. Res. 83, 578-582.
38. Kachi S., Terada M. and Hashimoto H. (1998) Effects of armorphous and polymorphs of PF1022A, a new antinematode drug, on Angiostrongylus costaricensis in mice. Jpn.J. Pharmacol. 77, 235-245.
39. Klein R. D. and Geary T. G. (1997) Recombinant microorganisms as tools for high throughput screening for nonantibiotic compounds, J. Biomol. Screening 2, 41-49.
40. Krasnoperov V., Bittner M. A., Holz R. W., Chepurny O. and Petrenko A. G. (1999) Structural requirements for α-latrotoxin binding and α-latrotoxin-stimulated secretion. J. Biological Chem. 274, 3590-3596.
41. Kozak M. (1989) The scanning model for translation: An update. J. Cell Biol. 108, 229-241.
42. Kurachi S., Sze L. and Kurachi K. (1998) Improved Transfection of Hep G2 Cells using FuGENE™ 6 Transfection Reagent. Biochemica 3, 43-44.
43. Lämmli U. K. (1970) Cleavage of structural proteins during the assembly of the head of bacteriophage T4. Nature 227, 680-685.
44. Lowry O. H., Rosebrough N. J., Farr A. L. and Randall R. J. (1951) Protein measurement with the Folin phenol reagent. J. Biol. Chem. 193, 265-275.
45. Martin R. J. (1997) Modes of action of anthelmintic drugs. Veterinary J. 154, 11-34.
46. Martin R. J., Robertson A. P. and Bjorn H. (1997) Target sites of anthelmintics. Parasitol. 114, 111-124.
47. Martin R. J., Harthe A., Londershausen M. and Jeschke P. (1996) Anthelmintic actions of the cyclic depsipeptide PF1022A and its electrophysiological effects on muscle cells of Ascaris suum. Pestic. Sci. 48, 343-349.
48. McCormack J. G. and Cobbold P. H. (1991) Cellular calcium: a practical approach. The practical approach series, IRL press, Oxford, 30-42.
49. McMaster G. K. and Carmichael G. G. (1977) Analysis of single- and double-stranded nucleic acids on polyacrylamide and agarose gels by using glyoxal and acridine orange. Proc. Natl. Acad. Sci. 74, 4835-4838.
50. Nakada M. T., Haskell K. M., Ecker D. J., Stadel J. M. and Crooke S. T. (1989) Genetic regulation of beta 2-adrenergic receptors in 3T3-L1 fibroblasts. Biochem. J. 260, 53-59.
51. Pearson W. R. and Lipman D. J. (1988) Improved tools for biological sequence comparison. Proc. Natl. Acad. Sci., U.S.A. 85, 2444-2448.
52. Petrenko A. G. (1993) α-Latrotoxin receptor. Implications in nerve terminal function. FEBS Lett. 325, 81-85.
53. Plasterk R. H. (1996) The Tc1/mariner transposon family. Curr. Top Microbiol. Immunol. 204, 125-143.
54. Rahman M. A., Ashton A. C., Meunier F. A., Davletov B. A., Dolly J. O. and Ushkaryov Y. A. (1999) Norepinephrine exocytosis stimulated by α-latrotoxin requires both external and stored Ca²⁺ and is mediated by latrophilin, G proteins and phospholipase C. Phil. Trans. R. Soc. Lond. B 354, 379-386.
55. Rees S., Coote J., Stables J., Goodson S., Harris S. and Lee M. G. (1996) Bicistronic vector for the creation of stable mammalian cell lines that predisposes all antibiotic-resistant cells to express recombinant protein. BioTechniques 20, 102-112.
56. Sambrook J., Fritsch E. F. and Maniatis T. (1989) Molecular cloning. A laboratory handbook. Cold Spring Harbor Laboratory Press, New York, 7.43-7.45.
57. Samson-Himmelstjerna G. von, Harder A., Schnieder T., Kalbe J. and Mencke N. (2000) In vivo activities of the new anthelmintic depsipeptide PF 1022A. Parasitol. Res. 86, 194-199.
58. Sasaki T., Takagi M., Yaguchi T., Miyadoh S., Okada T. and Koyama M. (1992) A new anthelmintic cyclodepsipeptide, PF1022A. J. Antibiotics 45, 692-697.
59. Schlack P. and Kumpf W. (1926) Über eine neue Methode zur Ermittlung der Konstitution von Peptiden. Z. Physiol.Chemie Hoppe-Seyler 154, 125-170.
60. Schmitt-Wrede H.-P., Waldraff A., Krücken J., Harder A. and Wunderlich F. (1999) Characterization of a hexokinase encoding cDNA of the parasitic nematode Haemonchus contortus. Biochim. Biophys. Acta 1444, 439-444.
61. Seagar M., Leveque C., Charvin N., Marqueze B., Martin-Moutot N., Boudier J. A., Boudier J.-L., Shoji-Kasai Y., Sato K. and Takahashi M. (1999) Interactions between proteins implicated in exocytosis and voltage-gated calcium channels. Phil. Trans. R. Soc. Lond. B 354, 289-297.
62. Smith T. F. and Waterman M. S. (1981) Identification of common molecular subsequences. J. Mol. Biol. 147, 195-197.
63. Sugita S., Ichtchenko K., Khvotchev M. and Sudhof T. C. (1998) α-Latrotoxin receptor CIRL/latrophilin 1 (CL1) defines an unusual family of ubiquitous G-protein-linked receptors. J. Biological Chem. 273, 32715-32724.
64. Tagaki M., Sasaki T., Yaguchi T., Kodama Y., Okada T., Miyadoh S. and Koyama M. (1991) On a new cyclic depsipeptide, PF1022A with anthelmintic effects. Nippon Nogeikagaku Kaishi 65, 326 (Abstr.).
65. Terada M. (1992) Neuropharmacological mechanism of action of PF1022A, an antinematode anthelmintic with a new structure of cyclic depsipeptide, on Angiostrongylus cantonensis and isolated frog rectus. Jpn. J. Parasitol. 41, 1-18.
66. Terada M., Ishih A., Tungtrongchitr A., Sano M. and Shomura T. (1993) Effects of PF1022A on developing larvae of Angiostrongylus costaricensis in mice, with special reference to route, dose and formulation. Jpn. J. Parasitol. 42, 199-210.
67. Thomas A. P. and Delaville F. (1991) The use of fluorescent indicators for measurements of cytosolic-free calcium concentration in cell populations and single cells. IRL Press, 1-54.
68. Waller P. J. (1997) Anthelmintic resistance. Veterinary Parasitology 72, 391-412.
69. Waller P. J. (1998) International approaches to the concept of integrated control of nematode parasites of livestock. Veterinary Parasitology 73, 155-164.
70. Yang F., Moss L. G. and Phillips G. N. Jr. (1996a.) The molecular structure of green fluorescent protein. Nature Biotechnology 14, 1246-1251.
71. Yang T. T., Cheng L. Z. and Kain S. R. (1996b) Optimized codon usage and chromophore mutations provide enhanced sensitivity with the green fluorescent protein. Nucleic Acid Res. 24, 4592-4593.

1. Use of calcium channel transmembrane receptors from helminths for identifying substances with anthelmintic activity.

2. Use of calcium channel transmembrane receptors from arthropods for identifying substances with arthropodicidal activity.

3. Use of transmembrane receptors according to claim 1 or 2, characterized in that they are G-protein-coupled transmembrane receptors with seven transmembrane domains.

4. Use of transmembrane receptors according to any of claims 1 to 3, characterized in that they are transmembrane receptors of the secretin subfamily.

5. Use of transmembrane receptors according to any of claims 1, 3 or 4, characterized in that they are transmembrane receptors from nematodes.

6. Use of transmembrane receptors according to any of claims 2 to 4, characterized in that they are transmembrane receptors from acarina.

7. Use of transmembrane receptors according to any of claims 1 and 3 to 5, characterized in that the transmembrane receptor HC110-R from Haemonchus contortus is involved.

8. Use of transmembrane receptors according to any of claims 1 to 6, characterized in that they are homologous to the transmembrane receptor HC110-R.

9. Use of transmembrane receptors according to any of claims 1 to 8 for I dentifying calcium channel blockers.

10. Use of transmembrane receptors according to any of claims 1 to 8 for identifying calcium channel blockers, characterized in that they are alpha-latrotoxin-binding transmembrane receptors.

11. Use of alpha-latrotoxin as agonist of transmembrane receptors according to any of claims 1, 3 to 5 and 7.

12. Use of alpha-latrotoxin as nematicide.

13. Use of alpha-latrotoxin in methods for identifying compounds having nematicidal and/or arthropodicidal activity.

14. Method for obtaining the HC110-R receptor and proteins homologous thereto, comprising the expression of the polypeptide or fragments thereof in a prokaryotic or eukaryotic expression system.

15. Method according to claim 14, characterized in that a eukaryotic expression system is involved.

16. Method according to claim 15, characterized in that HEK 293 or COS7 cells are used for the expression.

17. Host cells which make transient expression of the receptors HC110-R and proteins homologous thereto possible.

18. Host cells which make stable expression of the receptors HC110-R and proteins homologous thereto possible.

19. Host cells according to claim 18, characterized in that they express aequorin.

20. Host cells according to claim 18, characterized in that they are of the stably transformed cell line with the deposit number DSM ACC2464.

21. Host cells according to claim 18 or 19, characterized in that they are of the stably transformed cell line with the deposit number DSM ACC2465.

22. Vectors for stable transformation of host cells according to any of claims 18 to 21 and for transient transformation of host cells according to claim 17.

23. Vectors for stable transformation of host cells according to claim 18 or 20, characterized in that the vector pMyc6×His is involved.

24. Method for identifying agonists and/or antagonists of calcium channel transmembrane receptors from helminths and/or arthropods, comprising the following steps:
a) bringing a host cell according to any of claims 17 to 21 or membranes thereof into contact with a chemical compound or a mixture of chemical compounds under conditions which permit interaction of a chemical compound with the polypeptide, and
b) determining the chemical compound which specifically binds to the polypeptide.

25. Method for finding compounds which alter the expression of calcium channel transmembrane receptors from helminths or arthropods, comprising the following steps:
a) bringing a host cell according to any of claims 17 to 21 into contact with a chemical compound or a mixture of chemical compounds,
b) determining the calcium channel transmembrane receptor concentration, and
c) determining the compound which specifically affects the expression of the polypeptide.

26. Method according to claim 24 or 25, characterized in that G-protein-coupled transmembrane receptors of the secretin subfamily or fragments thereof are involved.

27. Method according to claim 24 or 25, characterized in that the transmembrane receptor HC110-R or fragments thereof and proteins homologous thereto are involved.

28. Method according to any of claims 24, 26 and 27, characterized in that the test substance is brought into contact with the transmembrane receptor under conditions which permit interaction of the receptor molecules with the test substance, and then
a) binding of the test substance which has taken place is detected, and
b) the activity of the receptor molecule in the presence of the test substance and its activity in the absence of a test substance are compared.

29. Method according to any of claims 24 to 28, characterized in that a cell-based test system is used.

30. Method according to claim 29, characterized in that cells according to any of claims 17 to 21 are used.

31. Method according to any of claims 24 and 26 to 28, characterized in that a cell-free test system is used.

32. Method according to any of claims 24 and 26 to 31, characterized in that the interaction of a test substance with the transmembrane receptor is detected through the displacement of alpha-latrotoxin bound thereto.

33. Method according to any of claims 24 and 26 to 31, characterized in that the interaction of a test substance with the transmembrane receptor is detected through the displacement of nifedipine bound thereto.

34. Use of nifedipine in a method according to any of claims 24 and 26 to 31.

35. Substances identified in a method according to any of claims 24 to 34.

36. Use of substances according to claim 35 for producing a composition for controlling helminths and/or arthropods.

37. Use of modulators of the HC110-R receptor and proteins homologous thereto as anthelmintics and/or arthropodicides.

38. Use of DNA coding for the HC110-R receptor and DNA homologous thereto for producing transgenic invertebrates.

39. Transgenic invertebrates which comprise the HC110-R receptor or proteins homologous thereto.

40. Transgenic invertebrates according to claim 39, characterized in that they are Drosophila melanogaster and Caenorhabditis elegans.

41. Use of DNA oligonucleotides which specifically hybridize onto the DNA coding for the HC110-R receptor for detecting DNA derived from helminths.

42. Method for detecting DNA from helminths, characterized in that
a) DNA oligonucleotides which hybridize onto the DNA coding for the HC110-R receptor or strands complementary thereto or onto the 5′- or 3′-flanking regions thereof are made available,
b) the DNA oligonucleotides are brought into contact with a DNA-containing sample,
c) the hybridization of the DNA oligonucleotide is detected,
d) the detected sequence is sequenced, and
e) the sequence is compared with the DNA sequence coding for the HC110-R receptor.

43. Diagnostic test kit comprising a DNA sequence coding for the HC110-R receptor, or fragment thereof or DNA sequences homologous thereto.

44. Diagnostic test kit according to claim 43, characterized in that the DNA sequences are provided with a detectable marker.

45. Use of the HC110-R receptor or fragments thereof and proteins homologous thereto for producing vaccines.


SEQUENCE LISTING (26 sequences)

SEQ ID NO: 1 - DNA, 3539 bp, Haemonchus contortus
ttctggccgtcatcgccgttgatgctgtcgctttcagaacgtctcttctattactaattcgaaaattgacgtgaatgagcagttcggtttaataccaacatgaggaatgtatatattcttctgctatccgtcgttgggacgtttaccgtcgcagaagaattgccagctcaatcaaacagttccattgtagtatgcgaaggtggtacagccgaattggaatgtccccatggtatggtcatttcaatagctctggcaaattatggccgatattcggctcgggtttgctacgagaacgaggaactggacgatgtggtaccgatgactcaatgccataaccctaaaacgatgccgacccttaggaaaagttgtgatggacgtcgtgaatgtcattttgtggttggtaacgacttcttcgtacatgatccatgcccaggagtgaagaaataccttgaagtcacatatctgtgtgttgctgatgtgactaccacaacaaccactacgacgacgaccaccaccactaccactacatccaccacaacagaggtggaggatgatgttaaagaagatatgagcgcgaaatctgcaccatcgacgtgtgcagcgacttcgagaagaggaatcgaatggcctgcgaccatttctgggacgacggtgaaccggccatgtcccgaaggcacgagaggcacgtcctcatggaaatgttccgccgaggggctgtggtctgaacctggtccaaatacaatagagtgtcgcagtgattggaccattcagcggcaggatgctcttgaagagacgataaaagatcaggatgcaagcggaattccggagcttcttcgagcgatgacgtctgatacccgaagacctatggtagctggtgatttgccaaaacttctaaacattcttgacatcgttcaagatgttgttggcagagaagtgtgggcaaaaagtagccagaagttggtgaaccagcttattgtgaatgtcgtacataatgcccttcgagcgaaggaaatgtggcagaactggccgtcagtgaagcgtcaaactttcgccacaagacttttgaatggtgttgaacgagccatgacctcctcctcaacaactgtctattcgtctgagaactatgtgcaaccgttggtgatgacagagatgagcgaaagcattcggacttcttcgcaaccatcgaactacttcctattcccttcaatggccttgtgggctggtgaaaataacgtggatagcgttgacgttccaagggaagctctcgaaatgacaggattggaccgtgcccgagtctactatgcgtcttttgcgaacattggtgaggaaatggaacctccggtggagatatcagcaggatctgaacagaagccgactggactagaaagacgtcgccgaattgtttcacgtgtggtggccgtgtcgcttgttctcgatggcaaagtcataaggctaccaatacttcctaagccgatcatcatcacattccatcattaccctgaggctttacggcggatgtcctcacctgaatgttcttggtgggatactgaagacatgaagtggtctacaagtggatgctccctacaatcgcataattctactcataccgtatgcgcttgtagtcatatgactcactttgccgttttgatggattatgtcggtcatgagatttcctccgaggataatcagcttcttacatttctcacatataccggttgcacgctgtcaatagtctgccttactctgacattcttctgttttgttgtcttcatcaaaggaggaggcgatagggttttcattcataaaaatctctgcgcttctcttggtatagccgaactggtgttcctcgctggaatttggaggacagaagaaaagtttgaatgcggtatgatcgctggttgcctcctttatttctttctatcggctttaacctggatgttactcgagggctaccagctttaccagatgcttgttgaggtgttccctgcctcgcgacgtcgattcacctttttccttgttggctatggcctgccagctatcattactggagcagccgcttattacgacccgactggttttggaacacgcaatcattgctggttacgcaccgacaaccttttcattttattctttgtcgcaccagcagctgtgattttactgacgaatacgatgttcctgttcatgactatgtgcattgtatacagacattcgaagtatatcccctgtaggcatgctgcggataatggaggtgacataaggacttgggtcaaaggagcgatgggtctggtctgtctacttggtgtcacatggacttgtggccttctctggatcgatgatggccattcgatagtgatggcatacgcgtttaccattgcgaattcccttcagggtcttttcatctttgtcttccatgtgctctgctcagagaagatgcgttacgatattgcccgatggtgtggcaagcatggcctttcatgtatcagctctggctcccgtgatacctctagggatctacaaaaacgtggaactatgtcaccgtcggagaggagtggcagcgaattcatctatcccacatcggagaagatgcacacatctcctcgtggattggaatcctctctcagctcggcctatcctcaacaaccactcatccaccactatcagcgtcgtccaccgcaacagcctaatggaacttatgactacgcaacaattgcgtatggcgagatggtaccaggccatatgttaccacgcatggcctcctcgtttcctcatccaggtgttgccatccattatccctcattcgatctggattcctcctaccaaccccagatcttccatcatcgccctccaccagacttttctccaccaccgccacctcctgcccagggtacaacaccttcaaaagtaatccgtcccccatcatccaagatgagtgacgactcggcctattccgatggtggatcgtcgtccgtactcaccacggaagtcacaccgtctggagcgactgtgcttcgaatggatttgggcagaaatcaaccgcctaactattggcgaaatgtttgagcatgtgtccctgtaaatgtttcctcgagagagatttttgccgaggttgtgcgcttcagtcacggtttatgacgatccgggtgcttttgcaatcattttgatgtgttcgatttgttcttccttggtatccaagggctttcatcgtcttccggacattctgtcacttttctacactctaatgctccaactgtcgttaatatcagcatatcatcgtcacaattatgcgccattttttctcagattaccggtttcctttgtacatttgtggattgtagttgtagctaaactgtagctattcagaggtatttttgatctagtcgataatagcgcaatattgttttactaattactaagtactaattgtttttttaagtccgattcctgccttctatggaaaaattttgttttcgtttccatcccgtatcatacttttaggttgagatcaataaaaacgtaaaatttgaaaaaaaaaaaaaaaa

SEQ ID NO: 2 - protein, 986 aa, Haemonchus contortus
MRNVYILLLSVVGTFTVAEELPAQSNSSIVVCEGGTAELECPHGMVISIALANYGRYSARVCYENEELDDVVPMTQCHNPKTMPTLRKSCDGRRECHFVVGNDFFVHDPCPGVKKYLEVTYLCVADVTTTTTTTTTTTTTTTTSTTTEVEDDVKEDMSAKSAPSTCAATSRRGIEWPATISGTTVNRPCPEGTRGTSSWKCSAEGLWSEPGPNTIECRSDWTIQRQDALEETIKDQDASGIPELLRAMTSDTRRPMVAGDLPKLLNILDIVQDVVGREVWAKSSQKLVNQLIVNVVHNALRAKEMWQNWPSVKRQTFATRLLNGVERAMTSSSTTVYSSENYVQPLVMTEMSESIRTSSQPSNYFLFPSMALWAGENNVDSVDVPREALEMTGLDRARVYYASFANIGEEMEPPVEISAGSEQKPTGLERRRRIVSRVVAVSLVLDGKVIRLPILPKPIIITFHHYPEALRRMSSPECSWWDTEDMKWSTSGCSLQSHNSTHTVCACSHMTHFAVLMDYVGHEISSEDNQLLTFLTYTGCTLSIVCLTLTFFCFVVFIKGGGDRVFIHKNLCASLGIAELVFLAGIWRTEEKFECGMIAGCLLYFFLSALTWMLLEGYQLYQMLVEVFPASRRRFTFFLVGYGLPAIITGAAAYYDPTGFGTRNHCWLRTDNLFILFFVAPAAVILLTNTMFLFMTMCIVYRHSKYIPCRHAADNGGDIRTWVKGAMGLVCLLGVTWTCGLLWIDDGHSIVMAYAFTIANSLQGLFIFVFHVLCSEKMRYDIARWCGKHGLSCISSGSRDTSRDLQKRGTMSPSERSGSEFIYPTSEKMHTSPRGLESSLSSAYPQQPLIHHYQRRPPQQPNGTYDYATIAYGEMVPGHMLPRMASSFPHPGVAIHYPSFDLDSSYQPQIFHHRPPPDFSPPPPPPAQGTTPSKVIRPPSSKMSDDSAYSDGGSSSVLTTEVTPSGATVLRMDLGRNQPPNYWRNV

SEQ ID NO: 3 - DNA, 48 bp, Artificial
  Anchor primer
cuacuacuacuaggccacgcgtcgactagtacgggnngggnngggnng

SEQ ID NO: 4 - DNA, 20 bp, Artificial
  Nested primer
ccattcgattcctcttctcg

SEQ ID NO: 5 - DNA, 20 bp, Artificial
  Nested primer 2
gtcgatggtgcagatttcgc

SEQ ID NO: 6 - DNA, 32 bp, Artificial
  Short anchor primer
cuacuacuacuaggccacgcgtcgactagtac

SEQ ID NO: 7 - DNA, 37 bp, Artificial
  Oligo dT adapter primer
ggccacgcgtcgactagtacttttttttttttttttt

SEQ ID NO: 8 - DNA, 32 bp, Artificial; identical to SEQ ID NO: 6
  Universal adapter primer

SEQ ID NO: 9 - DNA, 20 bp, Artificial
  Sequence specific primer
tttgttcttccttggtatcc

SEQ ID NO: 10 - DNA, 20 bp, Artificial
  Short adapter primer
ggccacgcgtcgactagtac

SEQ ID NO: 11 - DNA, 19 bp, Artificial
  Nested primer 3
acactctaatctccaactg

SEQ ID NO: 12 - DNA, 33 bp, Artificial
  P84_ATG BamHI 5′-primer
ctgccggatcctcggtttaataccaacatgagg

SEQ ID NO: 13 - DNA, 31 bp, Artificial
  P3121_TGA HindIII 3′-Primer
gcactaagcttgactgaagcgcacaacctcg

SEQ ID NO: 14 - DNA, 33 bp, Artificial
  P1434_TGA HindIII 3′-Primer
ggctcaagcttatcagagaacaagcgacacggc

SEQ ID NO: 15 - DNA, 42 bp, Artificial
  P2486_ATG BamHI 5′-Primer
ctatcggatcccaacatggctggctcccgtgatacctctagg

SEQ ID NO: 16 - DNA, 34 bp, Artificial
  P83EGFP_ATG HindIII 5′-Primer
ggtagaagcttttcggtttaataccaacatgagg

SEQ ID NO: 17 - DNA, 31 bp, Artificial
  P3057EGFP_o.TGA SalI 3′-Primer
ctgtgtcgacaacatttcgccaatagttagg

SEQ ID NO: 18 - DNA, 29 bp, Artificial
  P117Mausb2AR XhoI 5′-Primer
tacctcgagctgctaacctgccagccatg

SEQ ID NO: 19 - DNA, 33 bp, Artificial
  P1349Mausb2AR EcoRI 3′-Primer
tgtagaattcttccttccttgggagtcaacgct

SEQ ID NO: 20 - DNA, 34 bp, Artificial
  P70HumM1Rez XhoI 5′-Primer
atatctcgagagccccacctagccaccatgaaca

SEQ ID NO: 21 - DNA, 31 bp, Artificial
  P1465HumM1Rez EcoRI 3′-Primer
gacgaattccattggcgggagggagtgcggt

SEQ ID NO: 22 - DNA, 32 bp, Artificial
  P83MycTag_ATG BamHI 5′-Primer
ataggatccttcggtttaataccaacatgagg

SEQ ID NO: 23 - DNA, 31 bp, Artificial
  P3058MycTag_o.TGA XbaI 3′-Primer
cctgtctagaaacatttcgccaatagttagg

SEQ ID NO: 24 - DNA, 33 bp, Artificial
  P83_ATGNotI-5′-Primer
atattgcggccgcttcggtttaataccaacatg

SEQ ID NO: 25 - DNA, 27 bp, Artificial
  pMycHis_TGABamHI-3′-Primer
cgcggatcctagaaggcacagtcgagg

SEQ ID NO: 26 - DNA, 20 bp, Artificial sequence
  Primer
ggtcaccgtcgtcccagaaa